(19) United States
(12) Patent Application Publication
(10) Pub. No.: US 2019/0224243 A1
(43) Pub. Date: Jul. 25, 2019

(54) Enhancement of the Beneficial Effects of Mesenchymal Stem Cell Treatment by the Caveolin-1 Scaffolding Domain Peptide and Subdomains

(71) Applicant: MUSC Foundation for Research Development, Charleston, SC (US)

(72) Inventors: Stanley Hoffman, Charleston, SC (US); Elena Tourkina, Charleston, SC (US); Nicoletta Del Papa, Milano (IT)

(21) Appl. No.: 16/257,303

(22) Filed: Jan. 25, 2019

Related U.S. Application Data
(60) Provisional application No. 62/621,621, filed on Jan. 25, 2018.

(51) Int. Cl. A61K 35/28; A61K 9/00; C12N 5/0775; C12N 5/077; A61P 43/00

(52) U.S. Cl. CPC A61K 35/28; A61K 9/0019; A61P 43/00; A61K 9/0053; C12N 5/0653; C12N 5/0663

(57) ABSTRACT

Disclosed are compositions and methods for the use of mesenchymal stem cells (MSCs) in combination with caveolin scaffolding domain (CSD) peptide to treat fibrosis.

### CROSS-REFERENCE TO RELATED APPLICATIONS

This application claims priority to and the benefit of U.S. Patent Application No. 62/621,621, filed Jan. 25, 2018, the entirety of which is incorporated herein by reference.

### STATEMENT REGARDING FEDERALLY SPONSORED RESEARCH OR DEVELOPMENT

This invention was made with government support under AR062078 awarded by the National Institutes of Health (NIH). The government has certain rights in the invention.

### BACKGROUND OF THE INVENTION

Systemic sclerosis (SSc) is a chronic connective tissue disease involving the skin and internal organs (Gabrielli et al., 2009, N Engl J Med, 360:1989-2003). Its principal pathophysiological manifestations are vasculopathy, autoimmunity, and extensive multi-organ fibrosis (Solomon et al., 2013, Eur Respir Rev, 22:6-19). It is one of the most common diseases that fall under interstitial lung diseases (ILD), a grouping of devastating diseases involving lung fibrosis (Herzog et al., 2014, Arthritis Rheumatol, 66:1968-1978). In ILD, stiffening of the lung (i.e. fibrosis) leads to progressive shortness of breath, a very poor quality of life, and death within a few years. A key feature of lung fibrosis demographics is that African Americans experience increased prevalence, earlier age of onset, increased probability of the more severe diffuse form of scleroderma, and increased mortality. Compared to Caucasian patients, African Americans with ILD exhibit significantly decreased lung function including reductions in forced expiratory volume, forced vital capacity, and diffusing capacity for carbon monoxide (Steen et al., 2012, Arthritis Rheum, 64:2986-2994; Bogatkevich et al., 2007, Arthritis Rheum, 56: 2432-2442; Lee et al., 2015, Fibrogenesis Tissue Repair, 8:11).

Although each of the 130 diseases that fall under ILD qualify as an orphan disease, taken in aggregate these diseases are a significant health problem (Boland et al., 2013, Palliat Med, 27:811-816; Lee et al., 2014, Respir Med, 108:955-967) with a cost of over $20 billion per year in treatment and lost productivity. When fibrotic diseases of other organs (e.g. heart, kidney, liver, bone marrow, skin, eyes, wounds, and irradiated tissue) and fibrotic diseases of the lung that do not fall under ILD (asthma, chronic obstructive pulmonary disease) are taken into account, then the potential value of the market for novel treatments for lung fibrosis increases at least ten-fold. As the population ages with a growing number of people wishing to remain active with a high quality of life, the potential market for a treatment for lung fibrosis will continue to grow rapidly.

Mesenchymal stromal/stem cells (MSCs) have been explored as a treatment for SSc as they exhibit many relevant functions, including immunomodulation, promotion of angiogenesis, and inhibition of fibrosis (Maria et al., 2017, Clin Rev Allergy Immunol, 52:234-259; Maria et al., 2016, J Autoimmun, 70:31-39). MSCs are non-hematopoietic multipotent progenitor cells, first isolated and identified from bone marrow (BM) aspirates (Friedenstein et al., 1976, Exp Hematol, 4:267-274) which can also be isolated from adipose tissue and other sources (Maria et al., 2017, Clin Rev Allergy Immunol, 52:234-259). Adipose-derived MSCs have gained attention because of their accessibility and ease of harvest. In comparison with other sources, adipose-derived MSCs are more easily cultured, grow more rapidly, and can be cultured for longer time before becoming senescent (Kern et al., 2006, Stem Cells, 24:1294-1301).

MSCs have three key features important in their isolation, characterization, and potential use in the treatment of human diseases: 1) Adherence to standard tissue culture plastic; 2) Specific surface antigens: MSCs are CD73+, CD90+, CD105+, and CD45−; and 3) MSCs can differentiate into cell types including adipocytes, osteocytes, chondrocytes, fibroblasts, and myocytes (Maria et al., 2017, Clin Rev Allergy Immunol, 52:234-259).

MSC-based therapy in SSc patients has been very limited. Three month following BM MSC infusion in a patient with severe refractory SSc, the patient's digital ulcers were significantly decreased (Christopeit et al., 2008, Leukemia, 22:1062-1064). Six months after injection, blood flow to the patient's hands and transcutaneous oxygen pressure were improved. The same team later reported four similar cases with improved limb cutaneous symptoms and no major adverse effects (Keyszer et al., 2011, Arthritis Rheum, 63:2540-2542). An SSc patient treated with autologous BM MSC had complete healing of acute gangrene in upper and lower limbs (Guiducci et al., 2010, Ann Intern Med, 153:650-654). Injection into the affected skin of SSc patients of adipose-derived MSCs along with hyaluronic acid decreased skin thickness (Scuderi et al., 2013, Cell Transplant, 22:779-795). Despite these promising observations, one limitation to the widespread adoption of this approach is that the beneficial effect is of limited duration, so painful cell injections need to be repeated.

Although MSC-based therapy in SSc is promising, studies also reveal that SSc MSCs have altered phenotypes compared with healthy MSCs. For example, SSc MSCs are defective in their ability to differentiate into osteoblasts and adipocytes, in their angiogenic potential, and in that they exhibit reduced telomerase activity and early senescence (Del Papa et al., 2006, Arthritis Rheum, 54:2605-2615; Cipriani et al., 2007, Arthritis Rheum, 56:1994-2004; Cipriani et al., 2013, Clin Exp Immunol, 173:195-206). Moreover, increased profibrotic signaling via TGFβ and a decrease in the anti-fibrotic regulatory protein caveolin-1 are observed in SSc MSCs (Vanneaux et al., 2013, BMJ Open, 3).

Caveolin-1 is a promising therapeutic target in fibrotic diseases. It is a master regulatory protein that binds to and thereby inhibits the function or promotes the turnover of kinases in several signaling cascades (Tourkina et al., 2005, J Biol Chem, 280:13879-13887; Couet et al., 1997, J Biol Chem, 272:6525-6533; Le Saux et al., 2008, Am J Physiol Lung Cell Mol Physiol, 295:L1007-L1017; Oka et al., 1997, J Biol Chem, 272:33416-33421; Razani et al., 2001, J Biol Chem, 276:6727-6738; Rybin et al., 1999, Circ Res, 84:980-988; Wang et al., 2008, Am J Respir Crit Care Med, 178:583-591). Caveolin-1 is underexpressed in several cell types including fibroblasts and monocytes in SSc patients and in animal models (Tourkina et al., 2005, J Biol Chem, 280:13879-13887; Lee et al., 2014, Front Pharmacol, 5:140; Lee et al., 2014, Am J Physiol Lung Cell Mol Physiol, 306:L736-L748; Del Galdo et al., 2008, Arthritis Rheum, 58:2854-2865; Kasper et al., 1998, Histochem Cell Biol, 109:41-48; Tourkina et al., 2010, Ann Rheum Dis, 69:1220-1226). This deficiency leads to Col I overexpression by fibroblasts, monocyte hypermigration toward several chemokines, and to the enhanced differentiation of monocytes into CD45+/Col I+/α-smooth muscle actin+ (ASMA+) fibroblastic cells (Tourkina et al., 2011, Fibrogenesis Tissue Repair, 4:15; Tourkina et al., 2005, J Biol Chem, 280:13879-13887; Reese et al., 2014, Front Pharmacol, 16:141; Lee et al., 2014, Front Pharmacol, 5:140; Tourkina et al., 2010, Ann Rheum Dis, 69:1220-1226). The effects of caveolin-1 deficiency in cells and in animals can be reversed using the caveolin-1 scaffolding domain peptide (CSD, amino acids 82-101 of caveolin-1) (Tourkina et al., 2008, Am J Physiol Lung Cell Mol Physiol, 294:L843-L861; Wang et al., 2006, J Exp Med, 203:2895-2906). CSD enters cells (Tahir et al., 2009, Cancer Biol Ther, 8:2286-2296; Tahir et al., 2008, Cancer Res, 68:731-739) and acts as a surrogate for full-length caveolin-1 by inhibiting kinases just like full-length caveolin-1 (Bucci et al., 2000, Nat Med, 6:1362-1367; Bernatchez et al., 2005, Proc Natl Acad Sci USA, 102:761-766). In addition to the profibrotic effects of low caveolin-1 and their reversal by CSD in vitro, low caveolin-1 is profibrotic in vivo. Lung, skin, and heart fibrosis are observed in caveolin-1 KO mice (DelGaldo et al., 2008, Arthritis Rheum, 58:2854-65; Cohen et al., 2003, Amer Jour of Cell Phys, 284:C457-74; Drab et al., 2001, Science, 293:2449-52; Razani et al., 2001, J Biol Chem, 276:38121-38).). CSD also inhibits fibrosis in vivo in lung, skin, and heart (Tourkina et al., 2011, Fibrogenesis Tissue Repair, 4:15; Reese et al, 2014, Frontiers in Pharma, 5: epub; Tourkina et al., 2008, Amer Journal of Lung Cell Mol Phys, 294:L843-61; Pleasant-Jenkins et al, 2017, Lab Invest, 97:370-382.).

Pirfenidone and nintedanib are the only two FDA-approved drugs for treating idiopathic pulmonary fibrosis (IPF) the most common cases of ILD. However, pirfenidone is only marginally effective. Interestingly, the cellular mechanisms of action of pirfenidone and CSD overlap. Both pirfenidone and CSD inhibit collagen expression by fibroblasts, monocyte/fibrocyte migration in vitro, and the accumulation of fibrocytes in target tissues in vivo (Tourkina et al., 2011, Fibrogenesis Tissue Repair, 4:15; Inomata et al., 2014, Respir Res, 15:epub; Conte et al., 2014, Jour of the Euro Fed for Pharm Sci, 58:epub; Oku et al., 2008, Euro Jour of Phamr, 590:400-8; Tourkina et al., 2005, J Biol Chem, 280:13879-87; Reese et al., 2014, Front Pharmacol, 16:141; Lee et al., 2014, Front Pharmacol, 5:140). However, CSD is 300-fold more effective than pirfenidone in inhibiting fibrosis in an animal model for ILD (Kakugawa et al., 2004, Eur Respir J, 24:57-65; Tourkina et al., 2008, Am J Physiol Lung Cell Mol Physiol, 294:L843-L861), working at 1 mg/kg/day vs 300 mg/kg/day for pirfenidone.

Clearly, the development of CSD-based treatments for fibrosis is justified and is likely to be valuable for patients. Similarly, there is a need in the art for compositions and methods for enhancing the beneficial effect of MSC treatment on fibrotic tissue. The present invention satisfies this unmet need.

### SUMMARY OF THE INVENTION

The present invention provides compositions and methods for the use of mesenchymal stem cells (MSCs) in combination with caveolin-1 scaffolding domain (CSD) peptide or a subdomain or analog thereof to treat fibrosis.

In one aspect, the present invention provides a composition comprising a mesenchymal stem cell (MSC) and a caveolin-1 scaffolding domain (CSD) peptide or a subdomain, derivative, analog thereof. In various embodiments, the CSD peptide or a subdomain, derivative, analog thereof has an amino acid sequence of SEQ ID NO:1, SEQ ID NO:2, SEQ ID NO:3, SEQ ID NO:4, or any combination thereof. In one embodiment, the MSC has the ability to differentiate into an adipocyte. In one embodiment, the MSC is autologous, allogeneic, syngeneic, or xenogeneic to a subject having fibrosis. In one embodiment, the fibrosis is scleroderma.

In one aspect, the present invention provides a composition comprising a treated MSC. In one embodiment, the MSC is obtained after a treatment of culture with a CSD peptide, culture with a subdomain of a CSD peptide, genetic modification with a nucleic acid molecule encoding a CSD peptide, or genetic modification with a nucleic acid molecule encoding a subdomain of a CSD peptide. In various embodiments, the CSD peptide or a subdomain of a CSD peptide comprises an amino acid sequence of SEQ ID NO:1, SEQ ID NO:2, SEQ ID NO:3, SEQ ID NO:4, or any combination thereof. In one embodiment, the MSC has the ability to differentiate into an adipocyte. In one embodiment, the MSC is autologous, allogeneic, syngeneic, or xenogeneic to a subject having fibrosis. In one embodiment, the fibrosis is scleroderma.

In one aspect, the present invention provides a method of treating fibrosis. In one embodiment, the method comprises administering to a subject in need thereof an effective amount of a first composition comprising a MSC and an effective amount of a second composition comprising a CSD peptide or a subdomain, derivative, analog thereof. In various embodiments, the CSD peptide or a subdomain, derivative, analog thereof comprises an amino acid sequence of SEQ ID NO:1, SEQ ID NO:2, SEQ ID NO:3, SEQ ID NO:4, or any combination thereof. In one embodiment, the first composition and second composition are administered concurrently. In one embodiment, the first composition and second composition are administered at different times. In one embodiment, the MSC is treated with a composition of a CSD peptide, a subdomain of a CSD peptide, a nucleic acid molecule encoding a CSD peptide, or a nucleic acid molecule encoding a subdomain of a CSD peptide. In one embodiment, the MSC is treated prior to administration to the subject. In various embodiments, one or more of the first and second composition are administered to the subject locally, subcutaneously, intravenously, orally, intramuscularly, or any combination thereof. In one embodiment, the MSC differentiates into an adipocyte in the subject. In one embodiment, the MSC is autologous, allogeneic, syngeneic, or xenogeneic to a subject having fibrosis. In one embodiment, the fibrosis is scleroderma. In one embodiment, the subject is a human.

In one aspect, the present invention provides a method of treating fibrosis in a subject comprising administering to a subject in need thereof an effective amount of a composition comprising a MSC and a CSD peptide or a subdomain, derivative, analog thereof. In various embodiments, the CSD peptide or a subdomain thereof comprises an amino acid sequence of SEQ ID NO:1, SEQ ID NO:2, SEQ ID NO:3, SEQ ID NO:4, or any combination thereof. In various embodiments, the composition is administered to the subject locally, subcutaneously, intravenously, orally, intramuscularly, or any combination thereof. In one embodiment, the MSC differentiates into an adipocyte in the subject. In one embodiment, the MSC is autologous, allogeneic, syngeneic, or xenogeneic to a subject having fibrosis. In one embodiment, the fibrosis is scleroderma. In one embodiment, the subject is a human.

The present invention further includes the methods of treating fibrosis in a subject, the method comprising administering to a subject in need thereof an effective amount of a composition comprising a treated MSC. In one embodiment, the MSC is treated with a composition of a CSD peptide, a subdomain of a CSD peptide, a nucleic acid molecule encoding a CSD peptide, or a nucleic acid molecule encoding a subdomain of a CSD peptide. In one embodiment, the MSC is treated prior to administration to the subject. In one embodiment, the MSC differentiates into an adipocyte in the subject. In one embodiment, the MSC is autologous, allogeneic, syngeneic, or xenogeneic to a subject having fibrosis. In one embodiment, the fibrosis is scleroderma. In one embodiment, the subject is a human.

In one aspect, the present invention provides a kit comprising (a) a first composition comprising a MSC and (b) a second composition comprising a CSD peptide or a subdomain, derivative, or analog thereof. In one embodiment, the MSC is treated with a composition selected from the group consisting of a CSD peptide, a subdomain of a CSD peptide, a nucleic acid molecule encoding a CSD peptide, and a nucleic acid molecule encoding a subdomain of a CSD peptide.

### BRIEF DESCRIPTION OF THE DRAWINGS

The following description of exemplary embodiments of the invention will be better understood when read in conjunction with the appended drawings. It should be understood, that the invention is not limited to the precise arrangements and instrumentalities of the embodiments shown in the drawings.

FIG. 1, comprising FIG. 1A through FIG. 1F, depicts the results of exemplary experiments demonstrating the baseline differences between Healthy and Scleroderma (SSc) adipose tissue-derived (AT) mesenchymal stem cells (MSCs). AT MSCs from Healthy subjects, SSc patients without interstitial lung diseases (ILD), and SSc ILD patients were cultured in Maintenance Medium for 10 days, then analyzed by IHC or Western blotting. β-actin was used as the loading control for Western Blots. FIG. 1A depicts the results of exemplary experiments demonstrating caveolin-1 (Cav-1) levels in MSCs. Cav-1 levels were quantified by Western Blotting in extracts from four individuals in each group. Each symbol represents the data from one subject. p<0.001 for SSc No ILD vs SSc ILD. FIG. 1B depicts exemplary immunofluorescent images of Healthy and SSc ILD AT MSCs that were stained with the indicated antibodies and DAPI (nuclear stain). Representative results (n=4) are shown. FIG. 1C depicts exemplary extracts of Healthy and SSc ILD AT MSCs that were analyzed by Western Blotting. FIG. 1D depicts the results of exemplary experiments demonstrating Western blot data from four experiments as in FIG. 1C, that were quantified densitometrically using ImageJ. The level of each protein in Healthy AT MSCs was set to 100 Arbitrary Units. ***p<0.001; **p<0.01 for SSc ILD AT MSCs vs Healthy AT MSCs. FIG. 1E depicts exemplary extracts of late passage Healthy and SSc ILD AT MSCs that were analyzed by Western blotting. FIG. 1F depicts the results of exemplary experiments demonstrating Western blot data from four experiments as in FIG. 1E, that were quantified densitometrically using ImageJ. The level of each protein in Healthy AT MSCs was set to 100 Arbitrary Units.

FIG. 2, comprising FIG. 2A through FIG. 2C, depicts the results of exemplary experiments demonstrating the TGFβ and caveolin-1 siRNA treatment of Healthy AT MSCs. Treatments were performed as described in the Methods section. FIG. 2A depicts exemplary IHC images of ASMA staining. MSCs were treated with CSD, scrambled CSD (Scr), and TGFβ as indicated. Staining was quantified densitometrically in Arbitrary Units using ImageJ (n=4). The quantification is shown below the images. ***p<0.001 for TGFβ vs Control; \hat{ }\hat{ }\hat{ }p<0.001 for TGFβ+CSD vs TGFβ alone. FIG. 2B depicts exemplary extracts of Healthy AT MSCs treated with or without caveolin-1 siRNA that were analyzed by Western blotting with the indicated antibodies. FIG. 2C depicts the results of exemplary experiments demonstrating Western blot data from four experiments as in FIG. 2B, that were quantified densitometrically using ImageJ. The level of each protein in Control Healthy AT MSCs was set to 100 Arbitrary Units. ***p<0.001, *p<0.05 for caveolin-1 siRNA-treated MSCs vs Control MSCs.

FIG. 3, comprising FIGS. 3A and 3B, depicts the results of exemplary experiments demonstrating the TGFβ and adipocyte induction of Healthy AT MSCs. Treatments were performed as described in the Methods section. FIG. 3A depicts the exemplary extracts of Healthy AT MSCs receiving the indicated treatments (±Induction±TGFβ) that were analyzed by Western blotting with the indicated antibodies. FIG. 3B depicts the results of exemplary experiments demonstrating Western blot data from three experiments as in FIG. 3A, that were quantified densitometrically using ImageJ. The level of each protein in Uninduced cultures without TGFβ was set to 100 Arbitrary Units except for FABP4 and PPARγ for which the level in induced cultures without TGFβ was set to 100 Arbitrary Units. ***p<0.001, **p<0.01 for Uninduced+TGFβ vs Uninduced alone and Induced+TGFβ vs Induced alone; \hat{ }\hat{ }\hat{ }p<0.001 for Induced alone vs Uninduced alone.

FIG. 4, comprising FIG. 4A through FIG. 4D, depicts the results of exemplary experiments demonstrating the altered adipocyte differentiation of SSc AT MSCs. Healthy and SSc AT MSCs were induced for adipocyte differentiation±CSD and the scrambled control peptide (Scr) as described in the Methods section. FIG. 4A depicts exemplary IHC images of ASMA levels in the indicated cultures. FIG. 4B depicts the results of exemplary experiments demonstrating ASMA levels from three experiments as in FIG. 4A, that were quantified densitometrically using ImageJ. FIG. 4C depicts exemplary IHC images of FABP4 levels in the indicated cultures. FIG. 4D depicts the results of exemplary experiments demonstrating FABP4 levels from three experiments as in FIG. 4C, that were quantified densitometrically using ImageJ. FIG. 4E depicts the exemplary results from Oil Red 0-stained induced cultures of Healthy and SSc AT MSCs. ***p<0.001, *p<0.05 for Healthy+CSD vs Healthy and SSc+CSD vs SSc. \hat{ }\hat{ }\hat{ }p<0.001 for SSc vs Healthy.

FIG. 5, comprising FIG. 5A through FIG. 5D, depicts the results of exemplary experiments demonstrating the effects of TGFβ and CSD on Healthy MSCs already having undergone adipocyte differentiation. Healthy AT MSCs were induced to differentiate into adipocytes, then treated±TGFβ±CSD as described in the Methods section. FIG. 5A depicts exemplary extracts of the indicated cultures that were analyzed by Western blotting with the indicated antibodies. FIG. 5B depicts the results of exemplary experiments demonstrating Western blot data from three experiments as in FIG. 5A, that were quantified densitometrically using ImageJ. The level of each protein in cultures that did not receive TGFβ or CSD was set to 100 Arbitrary Units. ***p<0.001, **p<0.01 for CSD vs Control and CSD+TGFβ vs TGFβ alone. \hat{ }\hat{ }\hat{ }p<0.001, \hat{ }\hat{ }p<0.01, \hat{ }p<0.05 for TGFβ alone vs Control alone. FIG. 5C depicts exemplary extracts of the indicated cultures that were analyzed by Western blotting with the indicated antibodies. FIG. 5D depicts the results of exemplary experiments demonstrating Western blot data from three experiments as in FIG. 5C, that were quantified densitometrically using ImageJ. The level of each protein in cultures that did not receive TGFβ or CSD was set to 100 Arbitrary Units. ***p<0.001, **p<0.01 for CSD vs Control and CSD+TGFβ vs TGFβ alone. \hat{ }\hat{ }\hat{ }p<0.001, \hat{ }\hat{ }p<0.01, \hat{ }p<0.05 for TGFβ alone vs Control alone.

FIG. 6, comprising FIG. 6A through 6D, depicts the results of exemplary experiments demonstrating the differences between AT MSCs from Healthy and Bleomycin-treated Mice. AT MSCs isolated from Saline-treated (Healthy) mice and Bleomycin-treated mice were cultured in Maintenance Medium for 10 days, then analyzed by IHC or Western blotting. β-actin was used as the loading control for Western Blots. Healthy (Saline) and Bleomycin AT MSCs were induced for adipocyte differentiation as described in the Methods section, then analyzed by IHC. FIG. 6A depicts exemplary Healthy and Bleomcyin AT MSCs that were stained with the indicated antibodies and DAPI (nuclear stain). Representative results (n=3) are shown. FIG. 6B depicts (left) the exemplary extracts of Healthy and Bleomycin AT MSCs that were analyzed by Western Blotting and (right) the results of exemplary experiments demonstrating Western blot data from three experiments that were quantified densitometrically using ImageJ. The level of each protein in Healthy AT MSCs was set to 100 Arbitrary Units. *p<0.05 for Bleomycin vs Healthy AT MSCs. FIG. 6C depicts exemplary Saline and Bleomcyin AT MSCs induced for adipocyte differentiation that were stained with the indicated antibodies and DAPI (nuclear stain). FIG. 6D depicts the results of exemplary experiments demonstrating the levels of the indicated proteins in three experiments as in FIG. 6C, that were quantified densitometrically using ImageJ. ***p<0.001, **p<0.01 for Bleomycin vs Saline AT MSCs induced for adipocyte differentiation.

FIG. 7, comprising FIG. 7A through FIG. 7C, depicts the results of exemplary experiments demonstrating the effect on skin fibrosis of combined MSC and CSD treatment. Mice were treated with saline or bleomycin using osmotic minipumps (Lee R. et al., 2014 Am. J. Physio. Lung Cell. Mol. Physio. 306:L736-748), then injected subcutaneously once with MSCs on day 12. Injected MSCs were isolated from saline-treated mice (Sal) or Bleomycin-treated mice (Bleo). At the same time as MSC injection, daily i.p. injections of CSD or vehicle began. Mice were sacrificed on day 26 (n=4 per group), skin tissue sections prepared, stained with Masson's Trichrome, and scored in terms of dermal thickness. FIG. 7A depicts the results of exemplary experiments of Saline-treated host mice. FIG. 7B depicts the results of exemplary experiments of Bleomycin-treated host mice. *p<0.05 vs mice receiving bleomycin but no therapeutic treatment. FIG. 7C depicts exemplary IHC images of skin sections from mice receiving the indicated treatments that were stained for FABP4 and with DAPI to detect nuclei. The data from three animals per group were quantified densitometrically using ImageJ and are shown below the images. The statistical significance of the data is presented in Table 1.

FIG. 8, comprising FIG. 8A through FIG. 8C, depicts the results of exemplary experiments demonstrating the effect on lung fibrosis of combined MSC and CSD treatment. Mice were treated with bleomycin, MSCs, and CSD and sacrificed on day 26 (n=4 per group) as in FIG. 7. Lung tissue sections were prepared, stained with Masson's Trichrome (n=4 per group), and scored by the Ashcroft method (Ashcroft T. et al., 1988, J. Clin. Pathol. 41:467-470). FIG. 8A depicts the results of exemplary experiments demonstrating bleomycin-treated host mice. *p<0.05 vs mice receiving bleomycin but no therapeutic treatment. FIG. 8B depicts exemplary IHC images of lung sections from mice receiving the indicated treatments that were stained for ASMA and with DAPI to detect nuclei. The data from three animals per group were quantified densitometrically using ImageJ and are shown below the images. The statistical significance of the data is presented in Table 1. FIG. 8C depicts exemplary IHC images of lung sections from mice receiving the indicated treatments that were stained for HSP47 and with DAPI to detect nuclei. The data from three animals per group were quantified densitometrically using ImageJ and are shown below the images. The statistical significance of the data is presented in Table 1.

FIG. 9 depicts the results from exemplary experiments demonstrating dose-dependent inhibition of monocyte migration by CSD subdomains. The results shown are summarized from eleven independent experiments. These results demonstrate that three distinct subdomains of CSD may have beneficial effects on fibrotic diseases.

### DETAILED DESCRIPTION

The present invention is based partly on the discovery that mesenchymal stem cells (MSCs) injected into a mouse model of scleroderma resulted in significant improvements in adipose layer and dermal thickness. Further, CSD treatment reversed the pro-fibrotic phenotype of isolated SSc MSCs. Therefore, in various embodiments, the invention relates to compositions comprising MSCs in combination with CSD or a subdomain or analog thereof and their use in the treatment of fibrosis.

In various embodiments, the compositions and methods of the invention can be used to treat a fibrotic disease or disorder. Fibrosis can occur in many tissues within the body, including, but not limited to, lungs, liver, heart, brain, joints, skin, and bone marrow. Non-limiting examples of fibrotic diseases and disorders that can treated using the compositions and methods described herein include, but are not limited to interstitial lung disease, idiopathic pulmonary fibrosis, lung fibrosis, asthma, chronic obstructive pulmonary disease (COPD), Raynaud's phenomenon, pulmonary fibrosis, cirrhosis, atrial fibrosis, endomyocardial fibrosis, arthrofibrosis, Crohn's Disease, mediastinal fibrosis, myelofibrosis, tubulointerstitial fibrosis, hepatic fibrosis, premacular fibrosis, retinal fibrosis, dermal fibrosis, wound-associated fibrosis, Peyronie's disease, nephrogenic systemic fibrosis, progressive massive fibrosis, retroperitoneal fibrosis, fibroma, scleroderma, and radiation-induced fibrosis especially due to radiation therapy.

The present invention provides compositions comprising isolated MSCs. In one embodiment, the cells of the invention are multipotent. In one embodiment, the cells of the invention are capable of differentiating into adipocytes. In one embodiment, the isolated MSCs are autologous MSCs isolated from a subject with fibrosis.

The present invention also includes methods of treating fibrosis in a subject comprising administering to the subject isolated MSCs. In one embodiment, the isolated MSCs are treated with CSD, or a subdomain or analog thereof, or a combination thereof. In one embodiment, the isolated MSCs are treated with CSD, or a subdomain or analog thereof, or a combination thereof, prior to administration. In one embodiment, the MSCs are co-cultured with CSD, or a subdomain or analog thereof, prior to administration.

In one embodiment, invention includes methods of treating fibrosis in a subject comprising administering to the subject MSCs in combination with CSD or a subdomain or analog thereof, or a combination thereof. In one embodiment, the method comprises administering treated MSCs. In one embodiment, the treated MSCs are treated with CSD, or a subdomain or analog thereof, or a combination thereof, prior to administration.

In another embodiment, CSD or a subdomain thereof can be administered either alone or in combination with MSCs to treat a desired disease. In some instances, the MSCs can be pre-treated with CSD or a subdomain thereof, or a combination thereof prior to administrating to the subject in need thereof.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, ±0.1%, less than ±0.1%, or any percentage therebetween from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “allogeneic” refers to a biological material derived from a genetically different individual of the same species as the individual into whom the material will be introduced.

As used here, “biocompatible” refers to any material, which, when implanted in a mammal, does not provoke an adverse response in the mammal. A biocompatible material, when introduced into a mammal is not toxic or injurious to that mammal, nor does it induce immunological rejection of the material in the mammal.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

As used herein “conditioned media” defines a medium in which a specific cell or population of cells have been cultured in, and then removed. While the cells were cultured in said medium, they secrete cellular factors that include, but are not limited to hormones, cytokines, extracellular matrix (ECM), proteins, vesicles, antibodies, and granules. The medium plus the cellular factors is the conditioned medium.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, adipose derived adult stromal cell or other such progenitor cell, that is not fully differentiated when incubated in the medium, develops into a cell with some or all of the characteristics of a differentiated cell.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Expandability” is used herein to refer to the capacity of a cell to proliferate, for example, to expand in number or in the case of a cell population to undergo population doublings.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft,” “autologous transplant,” “autologous implant” and “autologous graft.” A graft comprising cells from a genetically different individual of the same species is referred to herein by the following interchangeable terms: “allograft”, “allogeneic transplant,” “allogeneic implant” and “allogeneic graft.” A graft from an individual to his identical twin is referred to herein as an “isograft”, a “syngeneic transplant”, a “syngeneic implant” or a “syngeneic graft.” A “xenograft,” “xenogeneic transplant” or “xenogeneic implant” refers to a graft from one individual to another of a different species.

As used herein, the term “growth medium” is meant to refer to a culture medium that promotes growth of cells. A growth medium will generally contain animal serum. In some instances, the growth medium may not contain animal serum.

An “isolated cell” refers to a cell which has been separated from other components and/or cells which naturally accompany the isolated cell in a tissue or mammal.

As used herein, the “lineage” of a cell defines the heredity of the cell, i.e.; which cells it came from and what cells it can give rise to. The lineage of a cell places the cell within a hereditary scheme of development and differentiation.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multi-lineage stem cell is a myogenic cell and an adipogenic cell (both are of mesodermal origin, yet give rise to different tissues). Another example is a neurogenic cell (of ectodermal origin) and adipogenic cell (of mesodermal origin).

As used herein, a “passage” refers to a round of subculturing. Thus, when cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but, not limited to, the seeding density, substrate, medium, and time between passaging.

As used herein, a “multipotent cell” defines a less differentiated cell that can give rise to at least two distinct (genotypically and/or phenotypically) further differentiated progeny cells.

The terms “precursor cell,” “progenitor cell,” and “stem cell” are used interchangeably in the art and herein and refer either to a pluripotent, a multipotent, or a lineage-uncommitted, progenitor cell, which is potentially capable of an unlimited number of mitotic divisions to either renew itself or to produce progeny cells which will differentiate into the desired cell type. Unlike pluripotent or multipotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, hydrogels, 3-dimensional amorphous shapes, etc.

“Self-renewal” refers to the ability to produce replicate daughter stem cells having differentiation potential that is identical to those from which they arose. A similar term used in this context is “proliferation.”

As used herein, “stem cell” defines an undifferentiated cell that can produce itself and a further differentiated progeny cell.

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of fibrosis is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Fibrosis involves the overexpression of extracellular matrix (ECM) proteins, primarily collagen, by α-smooth muscle actin (ASMA)-positive cells. Caveolin-1 is a master regulator of collagen expression by fibroblasts in vitro and in vivo. A caveolin-1 scaffolding domain peptide (CSD) inhibits ASMA expression in MSCs from healthy and scleroderma (SSc) subjects. Administration of MSCs in combination with CSD peptide to bleomycin-treated mice resulted in beneficial changes in lung tissue morphology. Therefore, disclosed herein are compositions comprising MSCs and a CSD peptide or a subdomain, derivative, or analogue thereof, and methods of use of the compositions in treating fibrosis in a subject in need thereof.

### Compositions

In various embodiments, the present invention relates to compositions comprising MSCs, compositions comprising a CSD peptide, or subdomain thereof, and combinations thereof for use in methods of treating fibrosis.

Caveolin-1 Scaffolding Domain Peptide (CSD)

Caveolin-1 is the principal coat protein of caveolae. Caveolae were originally observed in electron microscopic images as flask-shaped invaginations in the plasma membrane. These cholesterol- and sphingolipid-rich organelles function in endocytosis, vesicular trafficking, and in the compartmentalization of specific signaling cascades. The caveolin family of caveolae coat proteins contains three members of which caveolin-1 and -2 are abundantly expressed in adipocytes, endothelial cells, and fibroblasts. Caveolins serve as scaffolds for signaling molecules including members of the MAP kinase family, isoforms of PKC, Akt, G proteins, Src-family kinases, and growth factor receptors. The ability of caveolin-1 to bind to a variety of kinases and thereby inhibit their activity has been mapped to a sequence known as the caveolin-1 scaffolding domain (CSD, amino acids 82-101 of caveolin-1). A peptide equivalent to the CSD has the ability to bind to kinases and inhibits their activity.

In various embodiments, the invention relates to compositions comprising a CSD peptide, or subdomain, derivative, or analogue thereof. In one embodiment, a CSD peptide comprises an amino acid sequence of amino acids 82-101 of caveolin-1. In one embodiment, the CSD peptide comprises an amino acid sequence of DGIWKASFTTFTVTKYWFYR (SEQ ID NO:1). In one embodiment, a subdomain of the CSD peptide comprises at least six consecutive amino acid residues of the CSD peptide. In one embodiment, the subdomain comprises an amino acid sequence selected from DGIWKASF (SEQ ID NO:2), SFTTFTVT (SEQ ID NO:3), and VTKYWFYR (SEQ ID NO:4).

In one embodiment, a composition for use in the methods of the invention comprises a nucleic acid molecule encoding a CSD peptide or subdomain thereof. In one embodiment, the nucleic acid molecule encodes an amino acid sequence of amino acids 82-101 of caveolin-1. In one embodiment, the nucleic acid molecule encodes an amino acid sequence of DGIWKASFTTFTVTKYWFYR (SEQ ID NO:1). In one embodiment, the nucleic acid molecule encodes a subdomain of a CSD peptide. In one embodiment, the subdomain comprises at least eight consecutive amino acid residues of amino acids 82-101 of caveolin-1. In one embodiment, the subdomain comprises an amino acid sequence selected from DGIWKASF (SEQ ID NO:2), SFTTFTVT (SEQ ID NO:3), and VTKYWFYR (SEQ ID NO:4).

It is understood and herein contemplated that there are a number of variations of the CSD, or a subdomain thereof, that can be used in the disclosed methods of treatment. Specifically contemplated herein are modifications or mutations made to the CSD, or a subdomain thereof, that do not inhibit target binding, but can aid the peptide in, for example, avoiding proteolysis. Modifications and mutations of the CSD, or a subdomain thereof, that can be made include those described in detail in U.S. Pat. No. 8,058,227 B2 which is incorporated herein in its entirety.

It is further understood that the CSD peptide, or subdomain thereof, may be modified to aid entry into a cell. Therefore, contemplated herein are any known modifications that can be made to the CSD, or a subdomain thereof, that can aid entry into a cell. For example, the Antennapedia internalization sequence can aid in the ability to cross the plasma membrane. Thus, contemplated herein are compositions comprising a fusion peptide comprising the CSD, or a subdomain thereof, linked to the Antennapedia internalization sequence, and in some instances linked to the Antennapedia internalization sequence at the C-terminal. Also contemplated here are modifications to CSD or a subdomain thereof that, based on empirical data, aid entry into a cell. In addition, contemplated herein are methods of treating fibrosis comprising contacting a subject or cells ex vivo prior to their introduction into a subject with a composition comprising a fusion peptide comprising a CSD, or a subdomain thereof, and the C-terminus of the Antennapedia internalization sequence.

It is understood and herein contemplated that CSD peptides treat fibrosis through the binding to caveolin-1 binding domains. It is further understood that any variants of CSD such as derivatives or analogues of CSD or agents capable of binding to a caveolin-1 target will also be effective in the treatment of fibrosis. The identity of such agents, analogues, or derivatives of CSD, or a subdomain thereof, can be determined by the ability to not inhibit target binding.

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-comprising amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides comprising peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

Mesenchymal Stem/Stromal Cells

Mesenchymal stem cells (MSCs) are multipotent stem cells with high capacity of self-renewal and expansion, with differentiation potential to various cells including osteoblasts, chondrocytes, adipocytes, endothelial cells, nerve cells, heart myocytes, hepatocytes and pancreatic cells. The present invention relates to compositions comprising MSCs and cells derived therefrom and methods of using such cells. MSCs are useful for tissue engineering, wound repair, in vivo and ex vivo tissue regeneration, tissue transplantation, and other methods that require cells that can differentiate into a variety of phenotypes and genotypes, or can support other cell types in vivo or in vitro.

The invention further provides methods using MSCs in combination with CSD or subdomains thereof. In one embodiment, the MSCs of the invention are allogeneic, xenogeneic or syngeneic MSCs to a subject having fibrosis. In one embodiment, the MSCs of the invention are autologous MSCs isolated from a subject to whom they are then later administered. In one embodiment, autologous stem cell transplantation may be beneficial in reducing the likelihood of complications (e.g., graft-versus-host disease).

In one embodiment, the composition of the invention comprises MSCs. In one embodiment, the composition of the invention comprises one or more cell types derived from MSCs. Cells derived from the MSCs include, but are not limited to cells that differentiate from, dedifferentiate from, propagate from, and are downstream progeny of the MSCs.

In one embodiment, the MSCs may differentiate into cells of two or more lineages, for example adipogenic, chondrogenic, cardiogenic, dermatogenic, hematopoetic, hemangiogenic, myogenic, nephrogenic, neurogenic, neuralgiagenic, urogenitogenic, osteogenic, pericardiogenic, peritoneogenic, pleurogenic, splanchogenic, and stromal developmental phenotypes. In one embodiment, the MSCs can differentiate into three or more different lineages. In one embodiment, the MSCs can differentiate into four or more lineages.

In one embodiment, the MSCs may differentiate into mesodermal tissues, such as mature adipose tissue, bone, various tissues of the heart (e.g., pericardium, epicardium, epimyocardium, myocardium, pericardium, valve tissue, etc.), dermal connective tissue, hemangial tissues (e.g., endocardium, vascular epithelium, etc.), hematopoetic tissue, muscle tissues (including skeletal muscles, cardiac muscles, smooth muscles, etc.), urogenital tissues (e.g., kidney, pronephros, meta- and meso-nephric ducts, metanephric diverticulum, ureters, renal pelvis, collecting tubules, epithelium of the female reproductive structures (particularly the oviducts, uterus, and vagina), mesodermal glandular tissues (e.g., adrenal cortex tissues), and stromal tissues (e.g., bone marrow). Of course, inasmuch as the MSC can retain potential to develop into a mature cell, it also can realize its developmental phenotypic potential by differentiating into an appropriate precursor cell (e.g., a preadipocyte, a premyocyte, a preosteocyte, etc.).

In another embodiment, the MSCs may differentiate into ectodermal tissues, such as neurogenic tissue, and neurogliagenic tissue.

In another embodiment, the MSCs may differentiate into endodermal tissues, such as pleurogenic tissue, and splanchnogenic tissue, and hepatogenic tissue, and pancreogenic tissue.

In another embodiment, the inventive MSCs can give rise to one or more cell lineages from one or more germ layers such as adipocytes (of mesodermal origin) and myogenic cells (of mesodermal origin).

In yet another embodiment, MSCs may dedifferentiate into developmentally immature cell types, include dedifferentiating into an immature cell type, include embryonic cells and fetal cells or embryonic-like and fetal-like cells.

### Methods of Obtaining and Culturing MSCs of the Invention

The MSCs of the invention can be obtained from any animal by any suitable method. A first step in any such method requires the isolation of MSCs from the source animal. The animal can be alive or dead, so long as the MSCs are viable. Typically, human MSCs are obtained from a living donor. In one embodiment, MSCs may be obtained from non-human animals.

MSCs of the invention can be isolated from a variety of different sources, including, but not limited to, bone marrow, adipose tissue, cord blood, umbilical cord tissue, placenta, tendon, and peripheral blood.

In one embodiment, adipose-derived MSCs can be isolated from lipoaspirate using methods known in the art. Exemplary methods for isolating MSCs from lipoaspirate are described in (Francis et al., 2010, Organogenesis, 6(1):11-14) which is incorporated herein in its entirety.

In one embodiment, the isolated MSCs are resuspended and can be washed (e.g. in PBS). Cells can be centrifuged and resuspended successive times to achieve a greater purity. In one embodiment, the isolated MSCs cells may be a heterogeneous population of cells which includes the MSCs of the invention. MSCs may be separated from other cells by methods that include, but are not limited to, cell sorting, size fractionation, granularity, density, molecularity, morphologically, and immunohistologically. In one embodiment, MSCs of the invention are separated from other cells by assaying the length of the telomere, as stem cells tend to have longer telomeres compared to differentiated cells. In another embodiment, MSCs of the invention are separated from other cells by assaying telomeric activity, as telomeric activity can serve as a stem-cell specific marker. In another embodiment, MSCs of the invention are separated from other cells immunohistochemically, for example, by panning, using magnetic beads, or affinity chromatography. In one embodiment, MSCs can be separated through positive selection of one or more of expressed CD73/5′-Nucleotidase, CD90/Thy1, and CD105/Endoglin located on the surface of the MSCs. In one embodiment, the MSCs of the invention lack the expression of the hematopoietic stem cell marker CD34, the common leukocyte antigen CD45, CD11b or CD14, CD79 alpha or CD19, and HLA Class II. In one embodiment, the MSCs are CD73+, CD90+, CD105+, and CD45−. Accordingly, separation of MSCs may be carried out through positive selection, negative selection, or depletion. Such methods are well known in the art.

The isolated MSCs can be expanded or cultured according to known methods. In one embodiment, the MSCs can be cultured in vitro to maintain a source of MSCs. In one embodiment, the MSCs can be induced to differentiate into a desired cell type.

The MSCs can be cultured and, if desired, assayed for number and viability, to assess the yield. In one embodiment, the stem cells are cultured without differentiation using standard cell culture media (e.g., DMEM, typically supplemented with 5-15% (e.g., 10%) serum (e.g., fetal bovine serum, horse serum, etc.). In one embodiment, the stem cells are passaged at least one time in such medium without differentiating, while still retaining their developmental phenotype. In one embodiment, the MSCs are passaged in vitro at least 2 times, at least 3 times, at least 4 times, at least 5 times or more than 5 times.

In one embodiment, all cells extracted from a sample are cultured. To culture the cells, the cells may be plated at a desired density, such as between about 100 cells/cm² to about 100,000 cells/cm² (such as about 500 cells/cm² to about 50,000 cells/cm², or between about 1,000 cells/cm² to about 20,000 cells/cm²).

In one embodiment the extracted cells are plated at a lower density (e.g., about 300 cells/cm²) to facilitate the clonal isolation of the MSCs. For example, after a few days, MSCs plated at such densities will proliferate (expand) into a clonal population of MSCs.

Any suitable method for cloning stem cell populations can be used to clone and expand a MSC population of the invention. The cloning and expanding methods include cultures of cells, or small aggregates of cells, physically picking and seeding into a separate plate (such as the well of a multi-well plate). Alternatively, the stem cells can be subcloned onto a multi-well plate at a statistical ratio for facilitating placing a single cell into each well (e.g., from about 0.1 to about 1 cell/well or even about 0.25 to about 0.5 cells/well, such as 0.5 cells/well). The MSCs can be cloned by plating them at low density (e.g., in a petri-dish or other suitable substrate) and isolating them from other cells using devices such as a cloning rings. Alternatively, where an irradiation source is available, clones can be obtained by permitting the cells to grow into a monolayer and then shielding one and irradiating the rest of cells within the monolayer. The surviving cell then will grow into a clonal population. Production of a clonal population can be expanded in any suitable culture medium, for example, an exemplary culture condition for cloning stem cells (such as the inventive stem cells or other stem cells) is about ⅔ F12 medium+20% serum (e.g. fetal bovine serum) and about ⅓ standard medium that has been conditioned with stromal cells, the relative proportions being determined volumetrically).

In any event, whether clonal or not, the isolated MSCs can be cultured in a specific inducing medium to induce the MSCs to differentiate. The MSCs give rise to cells of multiple lineages, including mesodermal, ectodermal and endodermal lineages, and combinations thereof. Thus, MSCs can be treated to differentiate into a variety of cell types.

The MSCs also can be induced to dedifferentiate into a developmentally more immature phenotype (e.g., a fetal or embryonic phenotype). Such an induction is achieved upon exposure of the MSCs to conditions that mimic those within fetuses and embryos. For example, the inventive MSCs, can be co-cultured with cells isolated from fetuses or embryos, or in the presence of fetal serum.

The MSCs of the invention can be induced to differentiate into a mesodermal, ectodermal, or an endodermal lineage by co-culturing the cells of the invention with mature cells from the respective germ layer, or precursors thereof.

In an embodiment, induction of the MSCs into specific cell types by co-culturing with differentiated mature cells includes, but is not limited to, myogenic differentiation induced by co-culturing the MSCs with myocytes or myocyte precursors. Induction of the MSCs into a neural lineage by co-culturing with neurons or neuronal precursors, and induction of the MSCs into an endodermal lineage, may occur by co-culturing with mature or precursor pancreatic cells or mature hepatocytes or their respective precursors.

Alternatively, the MSCs are cultured in a conditioned medium and induced to differentiate into a specific phenotype. Conditioned medium is medium which was cultured with a mature cell that provides cellular factors to the medium such as cytokines, growth factors, hormones, and extracellular matrix. For example, a medium that has been exposed to mature myocytes is used to culture and induce MSCs to differentiate into a myogenic lineage. Other examples of conditioned media inducing specific differentiation include, but are not limited to, culturing in a medium conditioned by exposure to heart valve cells to induce differentiation into heart valve tissue. In addition, MSCs are cultured in a medium conditioned by neurons to induce a neuronal lineage, or conditioned by hepatocytes to induce an endodermal lineage.

In some instances, the conditioned media can include exosomes. For example, in addition to secreting extracellular proteins such as growth factors, cultured cells also secrete extracellular vesicles known as microvesicles or exosomes. Once thought of as contaminating debris in cell culture, these secreted microvesicles that are also called exosomes are packed with protein and RNA cargos. Exosomes contain functional mRNA, miRNA, DNA, and protein molecules that can be taken up by target cells. Proteomic and genomic analysis of exosome cargo has revealed a broad range of signaling factors that are both cell type-specific as well as differentially regulated based on the secreting cells' environment. The genetic information contained in exosomes may influence or even direct the fate of the target cell, for example by triggering target cell activation, migration, growth, differentiation or de-differentiation, or by promoting apoptosis or necrosis.

For co-culture, it may be desirable for the MSCs and the desired other cells to be co-cultured under conditions in which the two cell types are in contact. This can be achieved, for example, by seeding the cells as a heterogeneous population of cells onto a suitable culture substrate. Alternatively, the MSCs can first be grown to confluence, which will serve as a substrate for the second desired cells to be cultured within the conditioned medium.

Other methods of inducing differentiation are known in the art and can be employed to induce the MSCs to give rise to cells having a mesodermal, ectodermal, or endodermal lineage. In one embodiment, such methods include culturing the cells in a differentiation medium comprising one or more factors that induce the MSCs to differentiation. In one embodiment, a differentiation medium for inducing MSC differentiation into adipocytes comprises one or more of indomethacin, insulin, dexamethasone, troglitazone and 3-isobutyl-1-methylxanthine (IBMX). In one embodiment, the differentiation medium for inducing MSC differentiation into adipocytes comprises indomethacin, insulin, dexamethasone, and troglitazone.

In one embodiment, the MSCs of the invention are cultured in the differentiating-inducing medium for an amount of time to induce differentiation of at least 20%, at least 30%, at least 40%, at least 50%, at least 60%, at least 70%, at least 80%, at least 90%, or over 90% of the MSCs. In various embodiments, the MSCs are cultured in the differentiating-inducing medium for at least 5 days, at least 6 days, at least 7 days, at least 8 days, at least 9 days, at least 10 days, at least 11 days, at least 12 days, or more than 12 days. The MSCs can be assayed to determine whether, in fact, they have acquired the desired lineage.

Methods to characterize differentiated cells that develop from the MSCs of the invention, include, but are not limited to, histological, morphological, biochemical and immunohistochemical methods, or using cell surface markers, or genetically or molecularly, or by identifying factors secreted by the differentiated cell, and by the inductive qualities of the differentiated MSCs.

In one embodiment, a population of MSCs comprises one or more additional co-cultured cell types. For example, cells that can be co-cultured with the MSCs include other types of stem cells, such as neural stem cells (NSC), hematopoetic stem cells (HPC, particularly CD34+ stem cells), embryonic stem cells (ESC) and mixtures thereof, osteoblasts, neurons, chondrocytes, myocytes, adipocytes and precursors thereof. In other embodiments, the population is substantially homogeneous, consisting essentially of the inventive MSCs.

It may be desirable to induce differentiation of the MSCs of the invention. Therefore, in one embodiment of the invention, the MSCs of the invention can be induced to differentiate prior to being introduced into the recipient by, for example, culturing the MSCs in a differentiating-inducing medium. In another embodiment, the MSCs of the invention are not induced to differentiate, but are introduced into the recipient as a substantially pure population of cells that may differentiate following introduction into the recipient.

In one embodiment of the present invention, the MSC of the present invention is autologous. That is, a cell of the invention is procured from a donor and returned to the same individual after selection and expansion of said cell, i.e., donor and recipient are the same individual. In another embodiment of the present invention, the MSCs of the present invention are allogeneic. That is, the MSC of the invention is procured from a donor but administered to a different individual, i.e., the donor and recipient are genetically different individuals.

MSCs Treated with CSD

In one embodiment, the MSCs of the invention are treated with a CSD peptide, a subdomain of a CSD peptide or analog thereof, or a combination thereof. In one embodiment the treatment comprises culturing a MSC in the presence of a CSD peptide, subdomain thereof, or analog thereof. In one embodiment, the MSCs are cultured in the presence of a concentration of CSD, a subdomain thereof, or an analog, or a combination thereof sufficient to decrease, inhibit or prevent a pro-fibrotic phenotype in the MSC. In one embodiment, a pro-fibrotic phenotype is associated with expression of a pro-fibrotic marker in the MSCs. In various embodiments, the pro-fibrotic marker may be one or more of ASMA, Col I, and HSP47. In one embodiment, a pro-fibrotic phenotype is associated with decreased or loss of expression of a marker in the MSCs. In one embodiment, a pro-fibrotic phenotype is associated with decreased or loss of caveolin-1 expression in the MSCs.

In one embodiment, the MSCs are cultured with a CSD peptide comprising an amino acid sequence of amino acid residues 82-101 of caveolin-1. In one embodiment, the CSD peptide comprises an amino acid sequence of DGIWKASFTTFTVTKYWFYR (SEQ ID NO:1). In one embodiment, the MSCs are cultured with a subdomain of a CSD peptide comprising at least six consecutive amino acid residues of amino acids 82-101 of caveolin-1. In one embodiment, the subdomain comprises an amino acid sequence selected from DGIWKASF (SEQ ID NO:2), SFTTFTVT (SEQ ID NO:3), and VTKYWFYR (SEQ ID NO:4). In one embodiment, the CSD peptide or subdomain thereof comprises a modification that can aid entry into the MSCs. Modifications that can be made to the CSD peptide or subdomain thereof are discussed in detail elsewhere herein.

In various embodiments, a concentration of CSD peptide, a subdomain thereof, or an analog thereof sufficient to decrease, inhibit or prevent a pro-fibrotic phenotype in an MSC may be at least 10 pM, at least 100 pM, at least 10 nM, at least 100 nM, at least 1 μM, at least 10 μM, at least 100 μM, at least 1 mM, at least 10 mM, at least 100 mM, at least 1 M or greater than 1 M. In various embodiments, a concentration of CSD, a subdomain thereof, or an analog thereof may be less than 10 nM, less than 100 nM, less than 1 μM, less than 10 μM, less than 100 μM, less than 1 mM, less than 10 mM, less than 100 mM, or less than 1 M.

### Genetic Modification

In one embodiment, the MSCs may be treated with a nucleic acid encoding a CSD, a subdomain thereof, or a combination thereof. In one embodiment, the MSCs can be genetically modified, e.g., to express exogenous nucleic acid sequences. Therefore, the invention provides a method of genetically modifying such cells and populations. In one embodiment, an exogenous nucleic acid sequence encodes a CSD peptide, a subdomain of a CSD peptide, or a combination thereof. Therefore, in various embodiments the invention provides an isolated population of genetically modified MSCs comprising nucleic acid molecules for expression of CSD, a subdomain of a CSD peptide, or a combination thereof, and methods of preparing and using genetically modified MSCs.

In one embodiment, the MSCs is exposed to a gene transfer vector comprising an exogenous nucleic acid sequence, such that the nucleic acid molecule is introduced into the cell under conditions appropriate for the exogenous nucleic acid sequence to be expressed within the cell. The exogenous nucleic acid sequence generally is an expression cassette, including a polynucleotide operably linked to a suitable promoter.

In the context of gene therapy, the cells of the invention can be treated with a nucleic acid molecule encoding a CSD peptide, a subdomain of a CSD peptide, or a combination thereof prior to delivery of the cells into the recipient. In some cases, such cell-based gene delivery can present significant advantages of other means of gene delivery, such as direct injection of an adenoviral gene delivery vector. Delivery of a therapeutic nucleic acid molecule that has been pre-inserted into cells avoids the problems associated with penetration of gene therapy vectors into desired cells in the recipient.

Accordingly, the invention provides the use of genetically modified cells that have been cultured according to the methods of the invention. Genetic modification may, for instance, result in the expression of an exogenous nucleic acid sequence. Such genetic modification may have therapeutic benefit. Alternatively, or in addition, the genetic modification may provide a means to track or identify the cells so-modified, for instance, after administration of a composition of the invention into an individual. Tracking a cell may include tracking migration, assimilation and survival of a transplanted genetically-modified cell. Genetic modification may also include at least a second nucleic acid sequence. A second nucleic acid sequence may encode, for instance, a selectable antibiotic-resistance gene or another selectable marker.

The cells of the invention may be genetically modified using any method known to the skilled artisan. See, for instance, Sambrook et al. (2001, Molecular Cloning: A Laboratory Manual, Cold Spring Harbor Laboratory Press, Cold Spring Harbor, New York). For example, a cell may be exposed to an expression vector comprising an exogenous nucleic acid sequence, such that the nucleic acid is introduced into the cell under conditions appropriate for the exogenous nucleic acid sequence to be expressed within the cell. In one embodiment, the polynucleotide can encode a CSD peptide, a subdomain of a CSD peptide, or a combination thereof. In one embodiment, the polynucleotide encodes a peptide having an amino acid sequence selected from DGIWKASFTTFTVTKYWFYR (SEQ ID NO:1), DGIWKASF (SEQ ID NO:2), SFTTFTVT (SEQ ID NO:3), VTKYWFYR (SEQ ID NO:4), and a combination thereof.

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. Shorter polynucleotides are commonly referred to as “oligonucleotides” or “probes” of single- or double-stranded DNA.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell, such as yeast or bacteria, a eukaryote, such as an animal or mammalian cell, or in a plant).

Nucleic acids can be included within vectors as cell transfection typically employs a vector. The term “vector,” refers to, e.g., a plasmid, virus, such as a viral vector, or other vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides in operable linkage with a nucleic acid, and expressing the transcribed encoded protein in cells in vitro, ex vivo or in vivo.

A vector generally contains at least an origin of replication for propagation in a cell. Control elements, including expression control elements, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

Vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo- and rhabdoviruses, togavirus (e.g., sindbis virus and semliki forest virus) and vesicular stomatitis virus (VSV) for introducing and directing expression of a polynucleotide or transgene in pluripotent stem cells or progeny thereof (e.g., differentiated cells).

Vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. For expression in mammalian cells, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

In accordance with the invention, there are provided MSCs and their progeny transfected with a nucleic acid or vector. Such transfected cells include but are not limited to a primary cell isolate, populations or pluralities of multipotent stem cells, cell cultures (e.g., passaged, established or immortalized cell line), as well as progeny cells thereof (e.g., a progeny of a transfected cell that is clonal with respect to the parent cell, or has acquired a marker or other characteristic of differentiation).

The nucleic acid or protein can be stably or transiently transfected (expressed) in the cell and progeny thereof. The cell(s) can be propagated and the introduced nucleic acid transcribed and protein expressed. A progeny of a transfected cell may not be identical to the parent cell, since there may be mutations that occur during replication.

Viral and non-viral vector means of delivery into MSCs, in vitro, in vivo and ex vivo are included. Introduction of compositions (e.g., nucleic acid and peptide) into the cells can be carried out by methods known in the art, such as osmotic shock (e.g., calcium phosphate), electroporation, microinjection, cell fusion, etc. Introduction of nucleic acid and/or peptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers.

A peptide can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

### Treatment Methods

The invention is based, in part, on the discovery that administration of MSCs in combination with CSD was able to reduce the fibrotic phenotype in a fibrotic mouse model.

Accordingly, the compositions of the present invention can be used to treat a fibrotic disease or disorder. Non-limiting examples of fibrotic diseases and disorders that can treated using the compositions and methods described herein include, but are not limited to interstitial lung disease, idiopathic pulmonary fibrosis, lung fibrosis, asthma, COPD, Raynaud's phenomenon, pulmonary fibrosis, cirrhosis, atrial fibrosis, endomyocardial fibrosis, arthrofibrosis, Crohn's Disease, mediastinal fibrosis, myelofibrosis, tubulointerstitial fibrosis, hepatic fibrosis, premacular fibrosis, retinal fibrosis, dermal fibrosis, wound-associated fibrosis, Peyronie's disease, nephrogenic systemic fibrosis, progressive massive fibrosis, retroperitoneal fibrosis, fibroma and scleroderma. It is understood by those of skill in the art that the term treating, as used herein, includes repairing, replacing, augmenting, improving, preventing occurrence or recurrence, rescuing, repopulating, or regenerating.

In one embodiment, MSCs are extracted from a donor and are used to elicit a therapeutic benefit when administered to a recipient. In one embodiment, the donor and the recipient are not the same individual. Therefore in one embodiment the MSCs administered to a subject are allogeneic, syngeneic or xenogeneic MSCs to the recipient. In one embodiment, the donor and the recipient are the same individual. Therefore in one embodiment, the MSCs administered to a subject are autologous MSCs.

The MSCs may be extracted in advance and stored in a cryopreserved fashion or they may be extracted at or around the time of defined need. In one embodiment, isolated MSCs of the invention can be purified and/or expanded prior to administration to a subject. As disclosed herein, the cells may be administered to the patient, or applied directly to a damaged tissue, or in proximity of a damaged tissue, without further processing or following additional procedures to further purify, modify, stimulate, or otherwise change the cells. For example, the cells obtained from a patient may be administered to a patient in need thereof without culturing the cells before administering them to the patient.

In one embodiment, the cells obtained from a patient may be cultured prior to being administered to a patient in need thereof. In one embodiment, the MSCs are cultured in vitro for at least one, at least two, at least three, at least four, at least 5, or more than 5 passages. In one embodiment, the MSCs are cultured with a CSD peptide or subdomain thereof.

In one embodiment, the cells obtained from a patient may be modified prior to being administered to a patient in need thereof. In one embodiment, the MSCs are genetically modified with nucleic acid molecule encoding a CSD peptide, a subdomain of a CSD peptide, or a combination thereof.

In one embodiment, a composition comprising a MSC of the invention is administered to a subject in combination with a composition comprising a CSD peptide, a subdomain of a CSD peptide, or a combination thereof. In one embodiment, a MSC and a CSD peptide, a subdomain of a CSD peptide, or a combination thereof are formulated and administered as a single composition. In one embodiment, a MSC and a CSD peptide, a subdomain of a CSD peptide, or a combination thereof are formulated as separate compositions (e.g., formulated as a first composition comprising a MSC and a second composition comprising a CSD peptide, a subdomain of a CSD peptide, or a combination thereof.) Two or more compositions may be administered to the subject in any order and in any suitable interval. For example, in certain embodiments, the two or more compositions are administered simultaneously or near simultaneously. In certain embodiments, the method comprises a staggered administration of the two or more compositions, where a first composition is administered and a second composition administered at some later time point. Any suitable interval of administration, or treatment regimen, which produces the desired therapeutic effect may be used.

In one embodiment, a composition comprising a MSC is administered to a subject prior to administration of one or more composition comprising a CSD or subdomain thereof. In one embodiment, a treatment regimen comprises administering a composition comprising a MSC at least one day, at least 2 days, at least 3 days, at least 4 days, at least 5 days, at least 6 days, at least 1 week, at least 2 weeks, at least 3 weeks, at least 4 weeks, at least one month, at least two months or more than two months prior to an administration of a composition comprising a CSD or subdomain thereof. In one embodiment, a treatment regimen comprises administering a composition comprising a MSC less than one day, less than 2 days, less than 3 days, less than 4 days, less than 5 days, less than 6 days, less than 1 week, less than 2 weeks, less than 3 weeks, less than 4 weeks, less than one month, less than two months or less than two months prior to administration of a composition comprising a CSD or subdomain thereof.

In one embodiment, a treatment regimen comprises administering a composition comprising a CSD or subdomain thereof at least once prior to administration of a composition comprising a MSC. In one embodiment, a treatment regimen comprises administering a composition comprising a CSD or subdomain thereof at least one day, at least 2 days, at least 3 days, at least 4 days, at least 5 days, at least 6 days, at least 1 week, at least 2 weeks, at least 3 weeks, at least 4 weeks, at least one month, at least two months or more than two months prior to an administration of a composition comprising a MSC. In one embodiment, a treatment regimen comprises administering a composition comprising a CSD or subdomain thereof less than one day, less than 2 days, less than 3 days, less than 4 days, less than 5 days, less than 6 days, less than 1 week, less than 2 weeks, less than 3 weeks, less than 4 weeks, less than one month, less than two months or less than two months prior to administration of a composition comprising a MSC.

In one embodiment, a treatment regimen may include a single administration of a composition comprising a MSC and multiple administrations of comprising a CSD or subdomain thereof. In one embodiment, a treatment regimen may include multiple administrations of a composition comprising a MSC and a single administration of comprising a CSD or subdomain thereof. In one embodiment, a treatment regimen may include multiple administrations of a composition comprising a MSC and multiple administrations of comprising a CSD or subdomain thereof.

Multiple administrations of at least one composition of the invention can occur sequentially over a period of time selected by the attending physician. The time course and number of occurrences of administration can be dictated by monitoring the fibrotic tissue or a surrogate endpoint. Methods of assessment of treatment course are within the skill of the art of an attending physician. In one embodiment, the effects of cell delivery therapy would be demonstrated by, but not limited to, one of the following clinical measures: decreased dermal layer thickness, and increased adipose thickness. Other clinical measures that can be used include vital capacity and diffusion capacity for CO, forced vital capacity, 6-minute walk test, dyspnea scored according by Modified Medical Research Center, total lung capacity, shortness of breath reported by patient and mean change in Bronchoalveolar lavage components, and the likes.

A determination of the need for treatment will typically be assessed by a history and physical exam consistent with the fibrotic disease or disorder at issue. Subjects with an identified need of therapy include those with diagnosed fibrosis. Causes of fibrosis include, but are not limited to, genetic disorders, autoimmune disorders, inflammation, damage resulting from injury or trauma, damage from radiation or oxidative free radicals, or exposure to an environmental or medical agent.

A subject in need of treatment according to the methods described herein will be diagnosed with a fibrotic disease or disorder. In one embodiment, the subject is an animal, including, but not limited to, mammals (e.g., horses, cows, dogs, cats, sheep, pigs, and humans), reptiles, and avians (e.g., chickens).

It should be recognized that methods of this invention can easily be practiced in conjunction with existing therapies to effectively treat or prevent disease. The methods, compositions, and devices of the invention can include concurrent or sequential treatment with non-biologic and/or biologic drugs. In one embodiment, a drug for use in combination with the method of the invention may be one of pirfenidone and nintedanib.

As disclosed elsewhere herein, MSCs and/or compositions of the invention may be applied by several routes including systemic administration (e.g., intravenous injection) or by direct administration of the MSCs to the site of intended benefit. Systemic administration, particularly by intravenous means, has the advantage of being minimally invasive relying on the natural ability of the MSCs to target the site of damage. However, MSCs of the invention may be administered using any known administration route, including, but not limited to local, subcutaneous, intravenous or intramuscular routes of administration. Cells may be injected in a single bolus, through a slow infusion, or through a staggered series of applications separated by several hours or, provided cells are appropriately stored, several days or weeks.

In one embodiment, the route of delivery includes intravenous delivery through a standard peripheral intravenous catheter, a central venous catheter, or a pulmonary artery catheter. In one embodiment, cells are administered to the patient as an intra-vessel bolus or timed infusion. In another embodiment, MSCs may be re-suspended in an artificial or natural medium (e.g., a hydrogel, biocompatible lattice or tissue scaffold) prior to be administered to the patient.

### Pharmaceutical Formulations and Administration

Administration of one or more compositions of the present invention to a subject may be carried out using known procedures, at dosages and for periods of time effective to prevent or treat fibrosis in the subject. An effective amount of the one or more therapeutic compositions necessary to achieve a therapeutic effect may vary according to factors such as the state of the disease or disorder in the subject, and the age, sex, and weight of the subject. The regimen of administration may affect what constitutes an effective amount.

The dosages of the one or more compositions may be proportionally increased or decreased as indicated by the exigencies of the therapeutic situation. One of ordinary skill in the art would be able to study the relevant factors and make the determination regarding the effective amount of the therapeutic compound without undue experimentation.

Actual dosage levels of the active ingredients in the one or more pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors including the activity of the particular one or more compositions employed, the time of administration, the rate of excretion of the one or more compositions, the duration of the treatment, other drugs, compounds or materials used in combination with the one or more compositions, the age, sex, weight, condition, general health and prior medical history of the subject being treated, and like factors well known in the medical arts.

A medical doctor, e.g., physician or veterinarian, having ordinary skill in the art may readily determine and prescribe the effective amount of the one or more pharmaceutical compositions required. For example, the physician or veterinarian could start doses of the one or more compounds of the invention employed in the pharmaceutical composition at levels lower than that required in order to achieve the desired therapeutic effect and gradually increase the dosage until the desired effect is achieved.

Typically, dosages which may be administered in a method of the invention to a subject range in amount from 0.5 ng to about 50 mg per kilogram of body weight of the subject, while the precise dosage administered will vary depending upon any number of factors, including but not limited to, the type of subject and type of disease state being treated, the age of the subject and the route of administration. In one embodiment, the dosage of the compound will vary from about 1 ng to about 10 mg per kilogram of body weight of the subject. In one embodiment, the dosage will vary from about 3 ng to about 1 mg per kilogram of body weight of the subject.

To improve bioavailability and reduce the complications associated with repeated injections, the present invention contemplates sustained delivery of the compositions of the invention alone or in combination with other medications. The sustained delivery in the present invention can be achieved through a number of different delivery systems, including but not limited to polymeric gels, colloidal systems including liposomes and nanoparticles, cyclodextrins, collagen shields, diffusion chambers, flexible carrier strips, and implants.

The compositions of the present invention can be in the form of ointments. Ointments have the benefit of providing prolonged drug contact time with a surface. Ointments will generally include a base comprised of, for example, white petrolatum and mineral oil, often with anhydrous lanolin, polyethylene-mineral oil gel, and other substances recognized by the formulation chemist as being non-irritating, which permit diffusion of the drug, and which retain activity of the medicament for a reasonable period of time under storage conditions.

Therapeutic amounts of a composition of the invention can be administered orally. For these oral dosage forms, the composition may be formulated with a pharmaceutically acceptable solid or liquid carrier. Solid form preparations include powders, tablets, pills, capsules, cachets, and dispersible granules. The concentration or effective amount of the composition to be administered per dosage is widely dependent on the actual composition. However, a total oral daily dosage normally ranges from about 50 mg to 30 g, and in certain embodiments from about 250 mg to 25 g. A solid carrier can be one or more substances which may also function as a diluent, a flavoring agent, a solubilizer, a lubricant, a suspending agent, a binder, a preservative, a tablet disintegrating aid, or an encapsulating material. Suitable carriers include magnesium carbonate, magnesium stearate, talc, sugar, lactose, pectin, dextrin, starch gelatin, tragacanth, methylcellulose, sodium carboxymethylcellulose, microcrystalline cellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills cachets, and lozenges can be used as solid dosage forms suitable for oral administration.

A composition may also be contained within an inert matrix for either direct application or injection into the subject. As one example of an inert matrix, liposomes may be prepared from dipalmitoyl phosphatidylcholine (DPPC), for example, prepared from egg phosphatidylcholine (PC) since this lipid has a low heat transition. Liposomes are made using standard procedures as known to one skilled in the art. A composition, in amounts ranging from nanogram to microgram quantities, is added to a solution of egg PC, and the lipophilic drug binds to the liposome.

A time-release drug delivery system may be employed to result in sustained release of the active agent (e.g., a CSD or subdomain thereof) over a period of time. The time-release formation may be in the form of a capsule of a polymer (e.g., polycaprolactone, poly(glycolic) acid, poly(lactic) acid, polyanhydride) or lipids that may be formulation as microspheres. The composition bound with liposomes may be applied directly, either in the form of drops or as an aqueous based cream, or may be injected. In a formulation for direct application, the drug is slowly released overtime as the liposome capsule degrades due to wear and tear. In a formulation for injection, the liposome capsule degrades due to cellular digestion. Both of these formulations provide advantages of a slow release drug delivery system, allowing the subject a constant exposure to the drug over time.

In a time-release formulation, the microsphere, capsule, liposome, etc. may contain a concentration of a composition that could be toxic if administered as a bolus dose. The time-release administration, however, is formulated so that the concentration released at any period of time does not exceed a toxic amount. This is accomplished, for example, through various formulations of the vehicle (coated or uncoated microsphere, coated or uncoated capsule, lipid or polymer components, unilamellar or multilamellar structure, and combinations of the above, etc.). Other variables may include the subject's pharmacokinetic-pharmacodynamic parameters (e.g., body mass, gender, plasma clearance rate, hepatic function, etc.). The formation and loading of microspheres, microcapsules, liposomes, etc. and their implantation are standard techniques known by one skilled in the art.

Multiple compositions of the invention may be administered simultaneously, separately or spaced out over a period of time so as to obtain the maximum efficacy of the combination; it being possible for each administration to vary in its duration from a rapid administration to a continuous perfusion. As a result, for the purposes of the present invention, the combinations are not exclusively limited to those which are obtained by physical association of the constituents, but also to those which permit a separate administration, which can be simultaneous or spaced out over a period of time.

The administration of a nucleic acid encoding a CSD peptide or subdomain thereof of the invention to the subject may be accomplished using gene therapy. Gene therapy is based on inserting a therapeutic gene into a cell by means of an ex vivo or an in vivo technique. Suitable vectors and methods have been described for genetic therapy in vitro or in vivo, and are known as expert on the matter; see, for example, Giordano, Nature Medicine 2 (1996), 534-539; Schaper, Circ. Res 79 (1996), 911-919; Anderson, Science 256 (1992), 808-813; Isner, Lancet 348 (1996), 370-374; Muhlhauser, Circ. Res 77 (1995), 1077-1086; Wang, Nature Medicine 2 (1996), 714-716; WO94/29469; WO97/00957 or Schaper, Current Opinion in Biotechnology 7 (1996), 635-640 and the references quoted therein. A polynucleotide, or a polynucleotide encoding a peptide of the invention, can be designed for direct insertion or by insertion through liposomes or viral vectors (for example, adenoviral or retroviral vectors) in the cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (Proc. Natl. Acad. Sci. USA, 88 (1991), 2726-2729). The standard methods for transfecting cells with recombining DNA are well known by an expert on the subject of molecular biology, see, for example, WO94/29469. Genetic therapy can be carried out by directly administering the recombining DNA molecule or the vector of the invention to a patient.

### Kits

The invention also includes a kit comprising one more compositions described herein. For example, in one embodiment, the kit comprises a (a) first composition comprising a MSC and (b) a second composition comprising a CSD peptide or a subdomain, derivative, or analog thereof. In certain embodiments, the CSD peptide or a subdomain thereof comprises an amino acid sequence of SEQ ID NO:1, SEQ ID NO:2, SEQ ID NO:3, SEQ ID NO:4, or any combination thereof. In certain embodiments, the MSC is treated with a composition selected from the group consisting of a CSD peptide, a subdomain of a CSD peptide, a nucleic acid molecule encoding a CSD peptide, and a nucleic acid molecule encoding a subdomain of a CSD peptide.

The kit may comprise formulations of a pharmaceutical composition comprising the MSC or CSD peptide or subdomain, derivative, or analog thereof. In an embodiment, this kit further comprises a (optionally sterile) pharmaceutically acceptable carrier suitable for dissolving or suspending the compositions of the kit or invention, for instance, prior to administering the composition to an individual. Optionally, the kit comprises an applicator for administering one or more of the compositions.

In certain embodiments, the kit comprises instructional material that describes, for instance, the method of administering the compositions as described elsewhere herein. For instance, in some embodiments, the instructional material describes administering the composition, or combinations thereof, to an individual as a therapeutic treatment or a non-treatment use as described elsewhere herein.

Instructional material may include a publication, a recording, a diagram, or any other medium of expression which can be used to communicate the usefulness of compositions described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: CSD Treatment Reverses the Fibrotic Differentiation and Promotes the Adipogenic Differentiation

Autologous MSC therapy for treatment of fibrosis has been shown to have limited benefit with the beneficial effect observed right after MSC injections into patient's skin diminishing with time, thus requiring repeated injections. Without being bound by a particular theory, it is hypothesized that the environment in fibrotic tissue drives MSCs towards fibrogenesis and away from adipogenesis.

In the experiments presented herein, MSCs from fibrotic donors are demonstrated to have a fibrotic phenotype correlated with low caveolin-1 and be deficient in their ability to differentiate into adipocytes. Further, CSD treatment reverses the fibrotic differentiation and promotes the adipogenic differentiation of MSCs in vitro.

The materials and methods used in this experimental example are now described.

Isolation and Culture of MSCs

MSCs were isolated from adipose tissue from Healthy and SSc donors using a lipoaspirate method. Culturing of MSCs was performed using growth medium.

Differentiation Conditions

MSCs from healthy and SSc donors were treated with adipocyte induction medium (AIM), from Lonza to induce adipogenesis.

Promotion of Profibrotic Phenotype

To induce MSCs or adipocytes into a profibrotic phenotype, the cells were cultured in the presence of TGFβ.

Migration Experiments

Migration experiments were performed using TGFβ-activated monocytes and 100 ng/ml of the chemokine SDF-1 as chemoattractant (Tourkina et al., 2011, Fibrogenesis Tissue Repair, 4:15). Cells were treated with the indicated concentrations of the indicated peptides. Cells that had migrated were counted in six high power fields per filter. Migration is expressed in terms of “Inhibition of Migration”. Each experiment included Control monocytes and the same monocytes treated with TGFβ which enhanced their rate of migration approximately three-fold. If a test peptide had no effect on TGFβ-enhanced migration, then it had an “Inhibition of Migration of 0.0; if it decreased migration down to the level observed in the absence of TGFβ, then it had an “Inhibition of Migration” of 1.0. Intermediate degrees of inhibition were quantified on a linear basis.

SSc Mouse Model

To create a mouse model for autologous MSC treatment, adipose-derived MSCs were isolated from C57/BL6 mice treated with bleomycin, expanded in vitro, then injected systemically via the retro-orbital sinus or subcutaneously into host C57/BL6 mice that had been treated with bleomycin.

Generating Fibrosis in Mice

The Pump Model (Lee R. et al., 2014, Am. J. Physio. Lung Cell. Mol. Physio. 306:L736-748) is used both to generate MSC donors and host mice receiving MSC therapy. Briefly; under isoflurane anesthesia, mini-osmotic pumps (ALZET 1007D; DURECT Corporation, Cupertino, Calif.) containing 100 μl of bleomycin (67 U/kg) or saline are implanted posterior to the scapulae of 8-10 week old C57BL/6 mice. Pumps deliver 0.5 μl/h for 7 days, then are removed by day 10 per the manufacturer's instructions.

Autologous MSC Injection and Immunohistochemistry Analysis

Mice were treated with bleomycin or vehicle using osmotic minipumps (Lee et al., 2014, Am J Physiol Lung Cell Mol Physiol, 306:L736-L748). Mice were injected with MSCs on day 14, sacrificed on day 28, and tissue sections prepared and stained with Masson's Trichrome. Tissue morphology was quantified by the Ashcroft Method (Ashcroft et al., 1988, J Clin Pathol, 41:467-70).

Isolation and Injection of MSCs

21 days after implantation of pumps containing saline or bleomycin, inguinal fat is collected, minced, and digested with five volumes of 1 mg/ml collagenase (37° C., 30 minutes). The digest is diluted with Maintenance Medium (DMEM/10% FBS), filtered through a 70 μm mesh, and cells collected by centrifugation (800 g, 10 min, RT). Erythrocytes are lysed using ACK lysis buffer, cells collected by centrifugation, and cultured overnight in Maintenance Medium. Unattached cells are removed, bound MSCs are further cultured for one week till near confluence, lifted, and injected subcutaneously (5×10⁵ cells in 0.15 ml PBS) into the lower back of host mice ten days after the hosts had been treated with bleomycin or saline.

Treatment of mice with CSD (amino acids 82-101 of caveolin-1 (DGIWKASFTTFTVTKYWFYR; SEQ ID NO: 1) with the C-terminal COOH amidated) began the day after MSC injection and continued daily i.p. for 14 days. CSD was dissolved in 100% DMSO at 20 mM, then diluted 100-fold with water prior to injection of 100 μl.

Mouse Sacrifice and Analysis

On day 24, under anesthesia the rib cage was opened, mice systemically perfused via the left ventricle with PBS, then further perfused with Z-Fix (Anatech). Fixed lung and skin tissue was removed, sectioned, and stained with Masson's Trichrome. Lung tissue morphology was evaluated by the Ashcroft Method (Ashcroft T. et al., 1988, J. Clin. Pathol. 41:467-470). Skin tissue morphology was evaluated in terms of the thickness of the dermis and the subdermal adipose layer. Tissue sections were also analyzed by IHC using routine methods (Lee R. et al., 2014, Am. J. Physio. Lung Cell. Mol. Physio. 306:L736-748; Lee R. et al., 2014, Frontiers in Pharma. 5:140; Lee R. et al., 2015, Fibrogen. Tissue Repair, 8:11) and appropriate antibodies.

Human MSCs

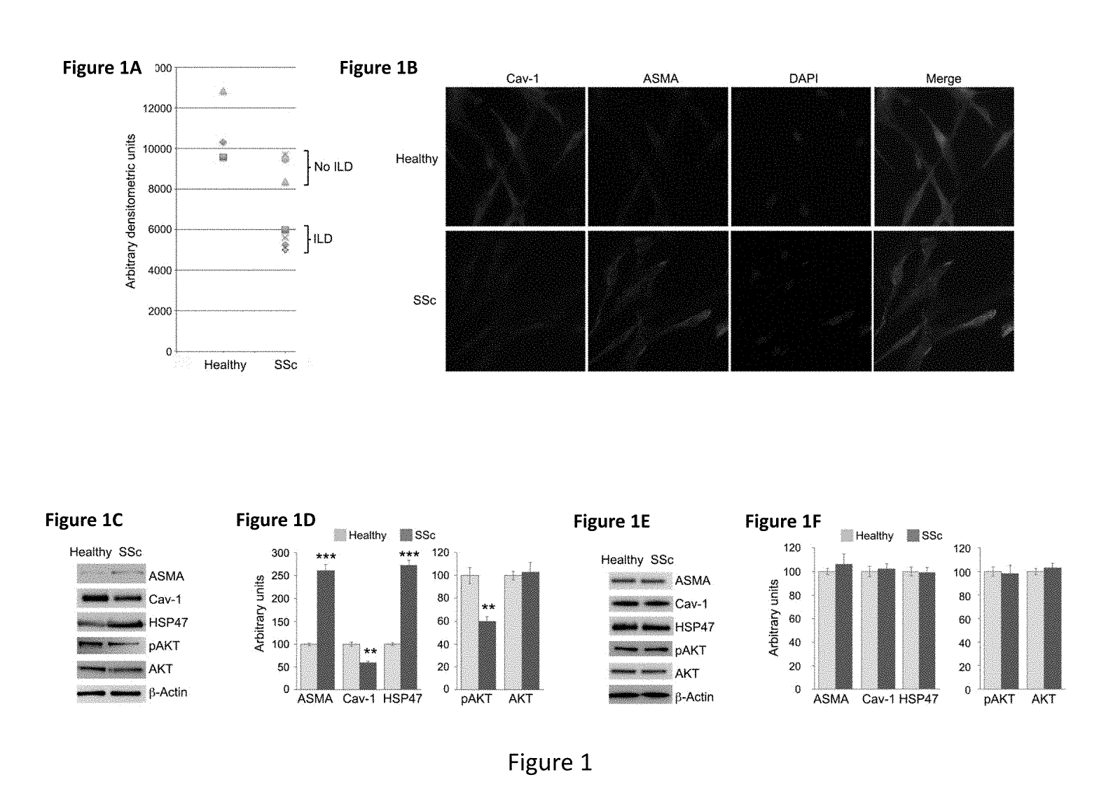

Human MSCs were derived from abdominal fat tissue. SSc ILD and SSc No ILD MSCs are a generous gift from Dr. Del Papa (Milan, Italy). Healthy human MSCs are from Dr. Del Papa and from Lonza. Human MSCs were maintained in the medium previously described (Capelli C. et al., 2017, Cell Transplant. 26:841-854). Early passage cells (passage 1 to 3) were used in all experiments except when late passage cells (passage 5) were specifically used in FIG. 1F.

MSC Differentiation and Analysis

To determine whether human or mouse MSCs exhibited fibrogenic differentiation at baseline, cells were incubated for 10 days in Maintenance Medium, then analyzed by IHC and/or Western blotting using appropriate antibodies. To induce fibrogenic differentiation in healthy human and mouse MSCs, cells were incubated for 10 days in Maintenance Medium supplemented with TGFβ (10 ng/ml). To determine the effects of knocking down caveolin-1 expression, healthy MSCs were treated with siRNA targeting caveolin-1 according to the manufacturer's instructions (Dharmacon).

Adipogenic differentiation was induced by incubating cells for 3 cycles of 3 days in Adipocyte Induction Medium (Lonza) plus 1 day in Maintenance Medium. Adipogenic differentiation was analyzed by IHC and/or Western blotting using appropriate antibodies and by staining with an Oil Red 0 kit (Biovision) according to the manufacturer's instructions.

Western Blotting

MSC cultures were rinsed with PBS. Lysis Buffer (20 mM Tris-HCl (pH 7.5)/1% NP-40/100 mM NaCl/5 mM EDTA/2 mM KCl) supplemented with 1 mM phenylmethylsulfonyl fluoride, protease inhibitor mixture Set V (Calbiochem), and phosphatase inhibitors (10 mM sodium pyrophosphate, 5 mM NaF, 10 mM beta-glycerophosphate, and 10 mM sodium orthovanadate) was added to the plates, the cells scraped into the buffer, frozen, thawed, and the extract clarified by centrifugation (10 min, 12,000 rpm, RT). The protein content of the supernatants was determined, sample buffer added, and the samples were boiled. 5 to 40 μg of protein was loaded per lane depending on the avidity of the primary antibody to be used for detection.

Statistical Analyses

Student's t test was used to analyze data comparing two samples. When comparing three or more groups, two-way analysis of variance (ANOVA) was used with the Bonferroni post-test. In experiments involving Western blots, immunoreactive bands were quantified by densitometry using Image J 1.32 NIH software. Raw densitometric data was analyzed using Prism 3.0 (GraphPad Software Inc.). Results were regarded as statistically significant if p<0.05.

The results of the experiments in this experimental example are now described.

### Baseline Differences Between Healthy and SSc AT MSCs

To determine the potential contribution of AT MSCs to the altered fibrogenesis and adipogenesis observed in SSc, several strains of AT MSCs from healthy subjects and SSc patients were compared as well as the role of caveolin-1 in regulating MSC differentiation. Caveolin-1 levels at early passage were first examined and showed that while MSCs from SSc ILD patients expressed significantly decreased levels of caveolin-1, MSCs from SSc patients without ILD exhibited intermediate levels (FIG. 1A). The examination of SSc ILD AT MSCs by IHC and Western blotting again demonstrated that these samples are deficient in caveolin-1, overexpress the myofibroblast marker ASMA and the collagen chaperone HSP47, and exhibit decreased AKT activation (FIG. 1B-FIG. 1D). FIG. 1 demonstrates that MSCs from healthy subjects contain relatively high levels of caveolin-1 and low levels of ASMA, while the converse is true for SSc-ILD MSCs. Given that SSc-ILD fibroblasts and monocytes contain relatively low levels of caveolin-1, it is noteworthy that SSc-ILD MSCs also contain only low levels of caveolin-1. The caveolin-1 deficiency is similar to what has been observed in fibroblasts and monocytes in SSc patients and linked to profibrotic behavior (Tourkina E. et al., 2005, J. Biol. Chem. 280:13879-13887; Tourkina E. et al., 2010 Annals of the Rheumatic Diseases, 69:1220-1226).

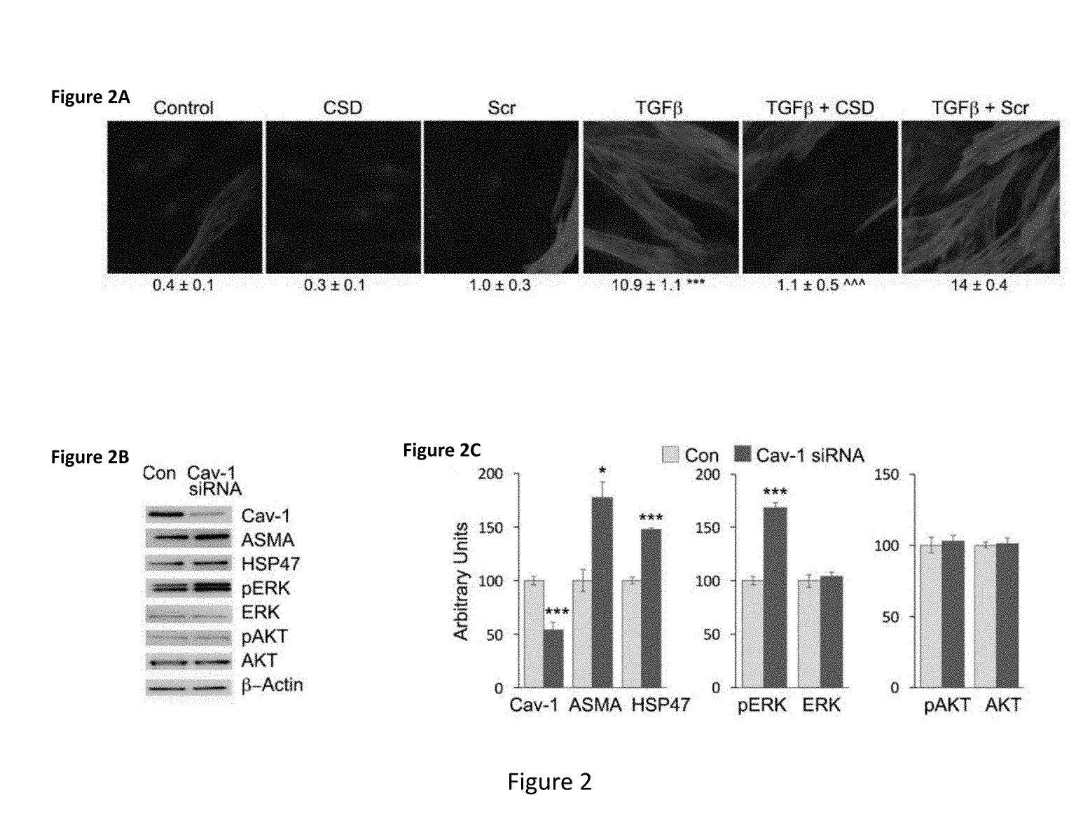

Although MSCs have been reported to be stable in phenotype in culture, at later passage these differences in caveolin-1, ASMA, HSP47, and AKT activation between healthy and SSc ILD AT MSCs were not observed (FIG. 1E and FIG. 1F), suggesting that the phenotype of SSc AT MSCs is altered in vivo due to the profibrotic environment and when cultured in vitro, they revert to a similar phenotype to healthy MSCs. To test this concept, healthy MSCs were treated in vitro with TGFβ. As predicted, this treatment enhanced ASMA expression (FIG. 2A). To further implicate caveolin-1 in the fibrogenic behavior of AT MSCs, siRNA were used to decrease caveolin-1 levels, and CSD were used to serve as a caveolin-1 surrogate. Indeed, in support of the profibrotic role of caveolin-1 depletion, specific siRNA decreased caveolin-1 levels; increased ASMA, HSP47 and pERK levels; while having no effect on pAKT (FIG. 2B and FIG. 2C). CSD inhibited both the baseline expression of ASMA in healthy MSCs and the enhanced expression observed in cells treated with TGFβ (FIG. 2A), while the scrambled control version of CSD did not differ from vehicle in its effect.

### Knock-Down of Caveolin-1 Mimics the Fibrogenic Phenotype of SSc MSCs

A knockdown of caveolin-1 in healthy MSCs resulted in the MSCs developing a pro-fibrotic phenotype, with increased levels of HSP47 and ASMA (FIG. 2B).

### Altered Induction of Adipogenesis in SSc AT MSCs

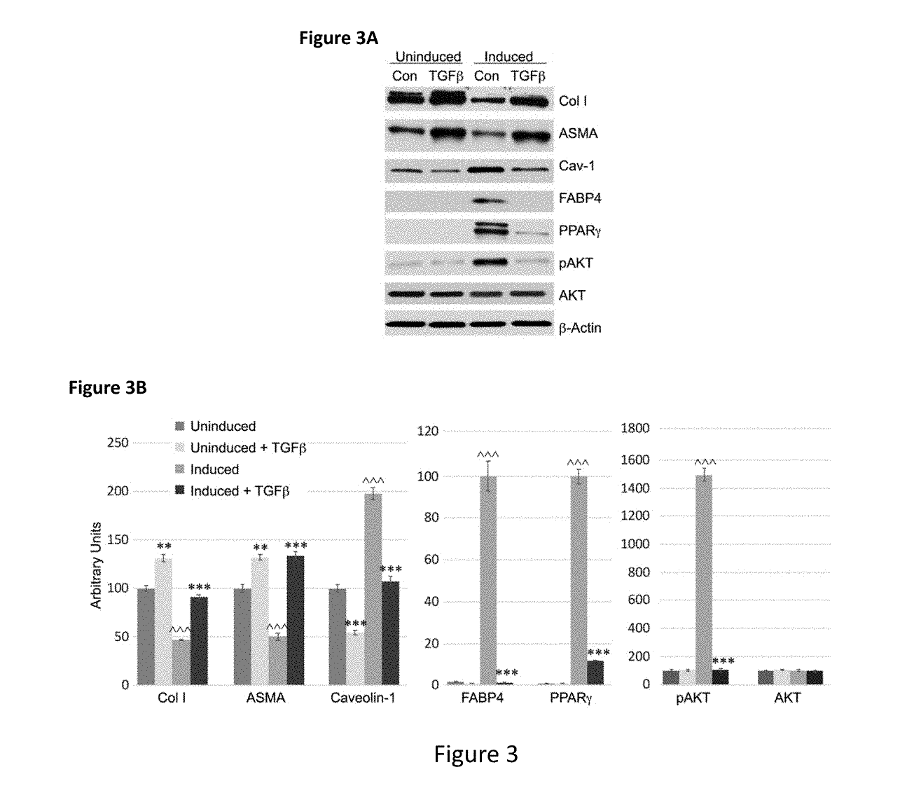

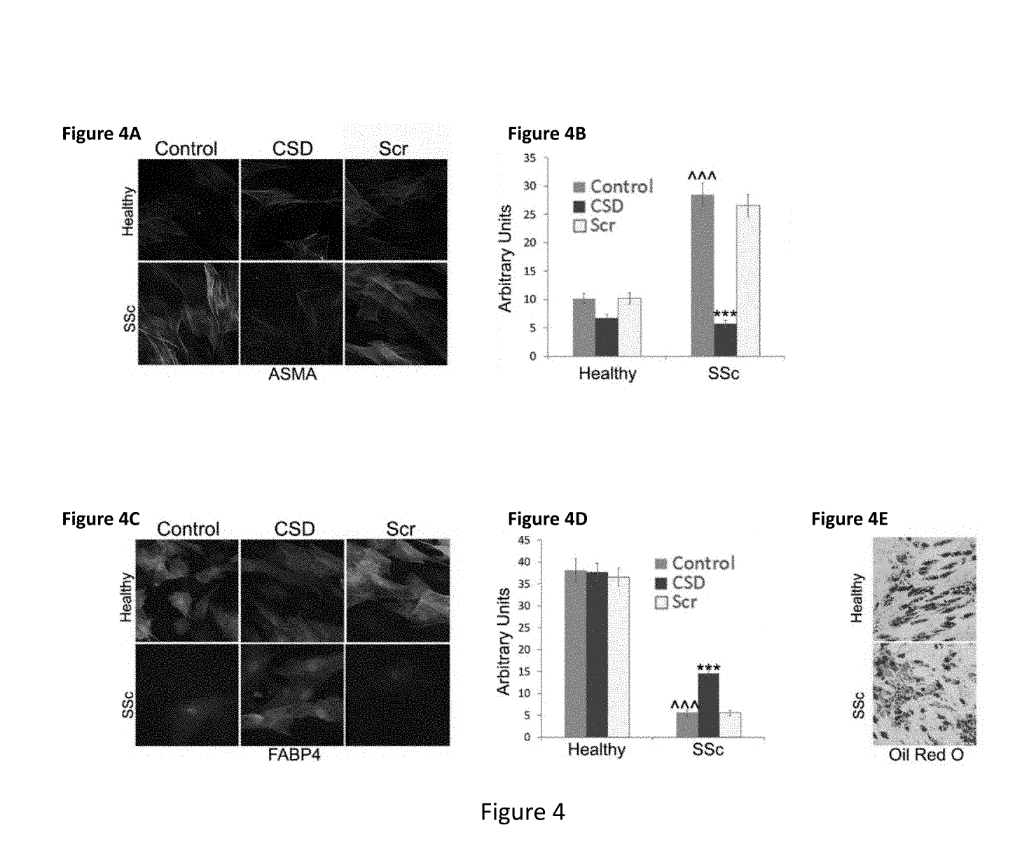

The present studies have demonstrated that induction results in increased levels of adipocyte markers, FABP4 and PPARγ. Further, induction increased AKT activation, which is known to be associated with adipogenesis (Peng X. D. et al., 2003, Genes Dev., 17:1352-1365) (FIG. 3A and FIG. 3B). Similarly, caveolin-1 levels increased, in accord with previous results (Lee R. et al., 2014, Frontiers in Pharma. 5:140) positively linking caveolin-1 levels and function to adipogenesis. In contrast, levels of myofibroblast markers ASMA and Col I decreased when adipogenesis was induced (FIG. 3A and FIG. 3B). The examination of the differentiation of SSc AT MSCs into adipocytes demonstrated that in contrast to Healthy AT MSCs, SSc AT MSCs responded poorly to induction in that few cells expressed FABP4 and the majority expressed ASMA (FIG. 4A-FIG. 4D). To demonstrate the role of caveolin-1 in the altered differentiation of SSc AT MSCs, the cells were treated with CSD during induction. As predicted, this treatment increased the number of FABP4+ cells and decreased the number of ASMA+ cells (FIG. 4A-FIG. 4D) while the scrambled control peptide had no effect. Although Healthy and SSc AT MSCs differed in FABP4 expression in response to induction, they did not differ in lipid droplet accumulation (FIG. 4E).

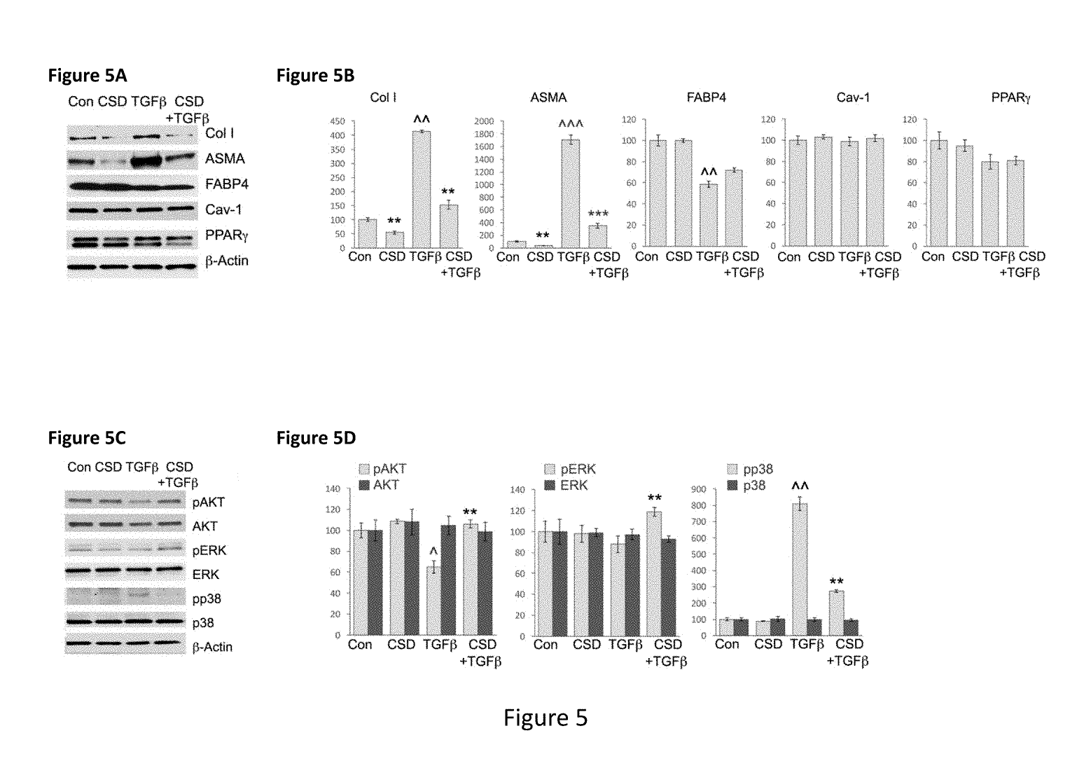

FIG. 4 demonstrates that healthy MSCs differentiate into adipocytes positive for the marker FABP4 with low levels of the fibrogenic marker ASMA when treated with adipocyte induction medium (AIM). When SSc-ILD MSCs are treated with AIM, they express ASMA at high levels, but are deficient in FABP4 (FIG. 4). When healthy MSCs are treated with both AIM and TGFβ, adipocyte differentiation is inhibited while fibrogenic differentiation occurs (FIG. 5A), consistent with the idea that fibrogenic differentiation and adipogenic differentiation are alternative fates of MSCs. The observations that TGFβ is profibrotic (increases Col I and ASMA, decreases caveolin-1) in both uninduced cells (cultured in growth medium) and cells treated with AIM, and that AIM induces adipocyte markers (FABP4 and PPARγ) whose levels are decreased by concomitant addition of TGFβ were confirmed by Western blot (FIG. 3A).

To model the onset of SSc, Healthy AT MSCs were differentiated into adipocytes, then the cells were treated with TGFβ. Even with this delayed treatment, TGFβ reversed adipogenesis (FABP4 expression) and promoted fibrogenesis (ASMA, Col I expression) (FIG. 5A and FIG. 5B). Consistent with the positive relationship between adipogenic potential and AKT activation (FIG. 1 and FIG. 3), TGFβ inhibited AKT activation (FIG. 5C and FIG. 5D), thus blocking adipogenesis and altering kinase activation (FIG. 5A-FIG. 5D). TGFβ also enhanced p38 activation (FIG. 5C and FIG. 5D).

Given that SSc-ILD MSCs are deficient in caveolin-1 and exhibit enhanced fibrogenic differentiation and inhibited adipogenic differentiation, without being bound by a particular theory, it was predicted that treatment with CSD (which serves as a surrogate for caveolin-1) would reverse the altered fibrogenesis and adipogenesis. Indeed this turned out to be the case for both fibrogenesis (FIG. 2A) and adipogenesis (FIG. 4 and FIG. 5A). CSD reversed the fibrogenic effect of TGFβ (increased ASMA and Col I expression), but did not restore FABP4 expression (FIG. 5A and FIG. 5B). Neither TGFβ nor CSD affected PPARγ or caveolin-1 expression (FIG. 5A and FIG. 5B). In contrast to effects of TGFβ on signaling, CSD inhibited p38 activation and restored AKT activation (FIG. 5C and FIG. 5D). Thus, TGFβ inhibits the adipogenesis of AT MSCs and this effect is reversed by CSD both during induction and when AT MSCs are already differentiated into adipocytes.

### AT MSCs in Murine Fibrosis Induced by Bleomycin

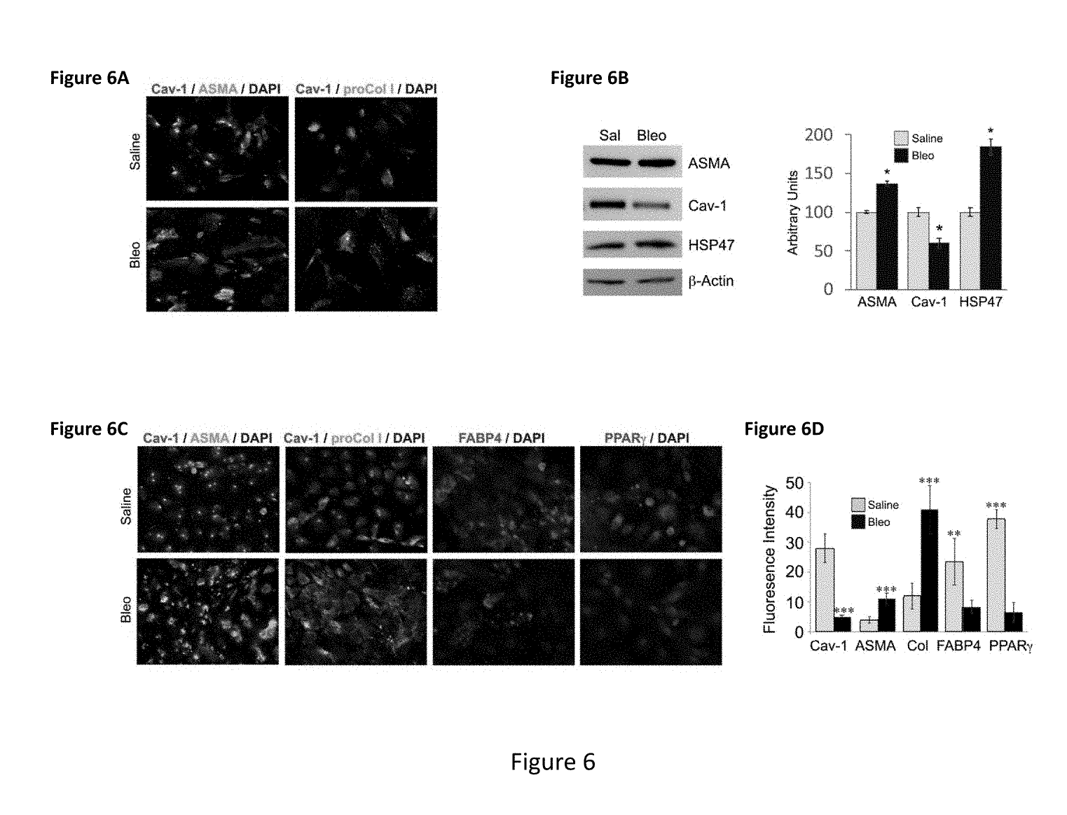

SSc is routinely modeled in mice via treatment with bleomycin delivered using subcutaneously-implanted osmotic minipumps (Lee R. et al., 2014, Am. J. Physio. Lung Cell. Mol. Physio. 306:L736-748). Besides inducing lung fibrosis, this treatment results in the thickening of the dermis and the thinning of the intradermal adipose layer (similar to what is observed in SSc patients (Fleischmajer R. et al., 1971, Science. 171:1019-1021)). Here, AT MSCs were successful isolated from bleomycin- and saline vehicle-treated mice and these cells were analyzed using the same approaches described above for human AT MSCs. Similar to the human data, AT MSCs from bleomycin-treated mice contained much less caveolin-1 and much more ASMA, HSP47, and Col I than AT MSCs from saline-treated mice (FIG. 6A and FIG. 6B). Experiments with MSCs isolated from fat tissue of healthy and bleomycin-treated mice validated the functional distinctions associated with fibrosis observed in human healthy and SSc-ILD MSCs. When AT MSCs were induced for adipogenesis, FABP4, PPARγ, and caveolin-1 were present at high levels in saline AT MSCs, but not in bleomycin AT MSCs (FIG. 6C and FIG. 6D). Conversely, ASMA and Col I were present at high levels in bleomycin AT MSCs, but not in saline AT MSCs. Thus, the behavior of AT MSCs from bleomycin-treated mice is very similar to the behavior of AT MSCs from SSc patients or AT MSCs from Healthy subjects that are treated with TGFβ after isolation.

The fibrogenic phenotype (low caveolin-1, high ASMA) can also be induced in MSCs from healthy adipose tissue by culturing healthy MSCs in the presence of TGFβ (FIG. 2A), a cytokine found at high levels in the blood and tissues of SSc patients. Similarly, MSCs from the adipose tissue of mice in which both lung and skin fibrosis was induced by systemic treatment with bleomycin also exhibit a fibrogenic phenotype (FIG. 6).

### Therapeutic Effects of MSC Injection

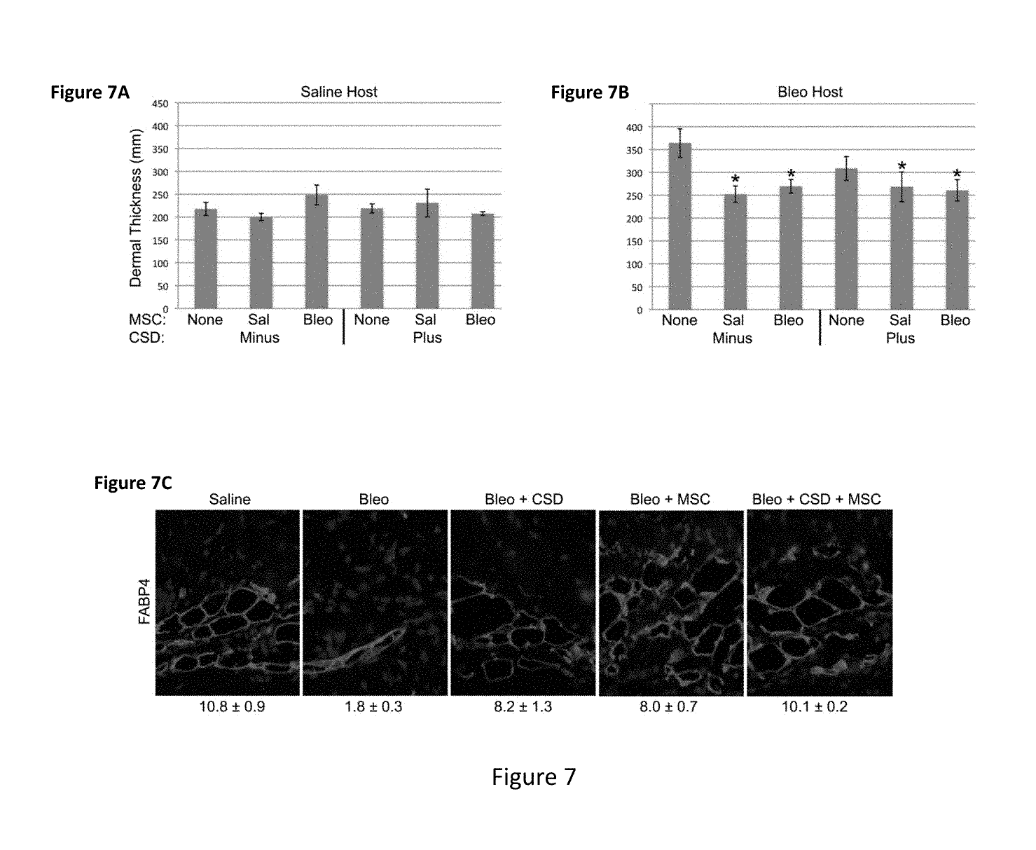

To evaluate the concept that MSC injection may have a beneficial effect on fibrosis, AT MSCs from saline- and bleomycin-treated donor mice were injected subcutaneously into saline and bleomycin-treated host mice. The dermal thickening that occurs in bleomycin-treated mice was significantly decreased by the injection of AT MSCs derived from either saline- or bleomycin-treated mice (FIG. 7A and FIG. 7B). FABP4 staining (FIG. 7C, quantified in Table 1) revealed a major loss of the intradermal adipose layer in bleomycin-treated mice that was reversed by either AT MSC injection or by CSD treatment and most effectively by combined treatment.

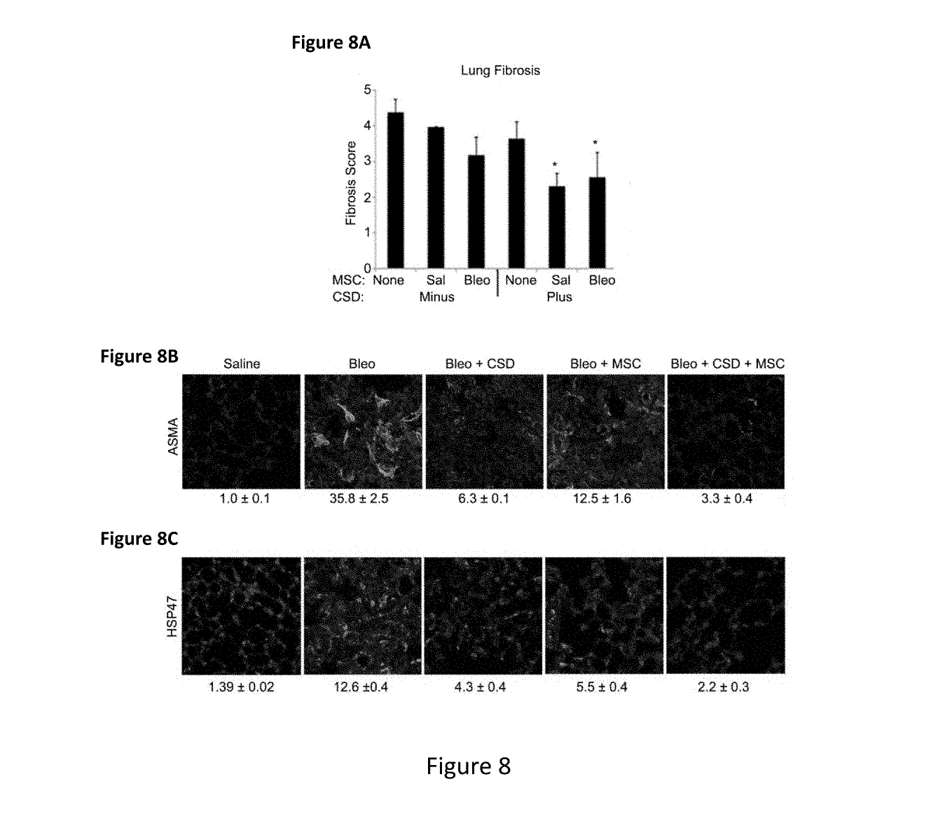

TABLE 1
p Values for IHC Data from FIG. 7 and FIG. 8. (IHC data were
quantified in terms of the percent of the field that was stained.
Data were obtained from at least three mice per condition, six
fields per mouse. For Bleo + MSC and Bleo + CSD +
MSC, no difference was observed between the beneficial
effects of the injection of MSCs from saline-treated and
bleomycin-treated mice, so the data were pooled.)

| | FIG. 7 | FIG. 8 | FIG. 8 |
|---|---|---|---|
| Comparison | Skin FABP4 | Lung ASMA | Lung HSP47 |
| Saline vs Bleo | p < 0.001 | p < 0.001 | p < 0.01 |
| Bleo vs Bleo + CSD | p < 0.01 | p < 0.001 | p < 0.001 |
| Bleo vs | p < 0.001 | p < 0.001 | p < 0.05 |
| Bleo + MSC | | | |
| Bleo + CSD vs | p < 0.05 | p < 0.05 | p < 0.01 |
| Bleo + CSD + MSC | | | |
| Bleo + MSC vs | p < 0.01 | p < 0.05 | p < 0.001 |
| Bleo + CSD + MSC | | | |

While AT MSC treatment alone had a beneficial effect on skin morphology and adipocyte markers (FIG. 7), it did not have a beneficial effect on lung morphology (FIG. 8). However, combined treatment of mice with MSCs and CSD did improve lung morphology (FIG. 8A). Moreover, the benefits of combined CSD-MSC treatment were validated by IHC for fibrosis markers ASMA and HSP47 as well as by the decrease in cellularity observed with dual treatment compared to either single treatment (FIG. 8B and FIG. 8C, quantified in Table 1). The beneficial effects of MSC treatment both on skin and lung fibrosis were similar whether these cells were derived from the fat of healthy mice or mice treated with bleomycin (FIG. 7 and FIG. 8).

Combination Therapy of Skin and Lung Fibrosis In Vivo with MSCs and CSD

Treatment of SSc patients with autologous adipose-derived MSCs has been beneficial in treating skin fibrosis, but the effect was not long-lasting. Whether there are potential beneficial effects of MSC treatment on lung fibrosis is still uncertain. CSD has been demonstrated to have beneficial effects on lung, skin, and heart fibrosis (Reese et al., 2014, Front Pharmacol, 16:141; Lee et al., 2014, Front Pharmacol, 5:140; Tourkina et al., 2008, Am J Physiol Lung Cell Mol Physiol, 294:L843-L861; Pleasant-Jenkins et al., 2017, Lab Invest, 97:370-382); however, this beneficial effect on lung morphology is observed only in slightly greater than 50% of mice receiving treatment (Reese et al., 2014, Front Pharmacol, 16:141).

MSC treatment provided beneficial effects on both skin and lung fibrosis (FIG. 7 and FIG. 8) in an SSc mouse model. Statistically significant beneficial effects on skin fibrosis were observed in reversing the thickening of the dermis (p<0.05). Equivalent results were obtained both with MSCs derived from the fat of healthy mice and of bleomycin-treated mice. No additional beneficial effect was observed when CSD treatment was combined with MSC treatment. In contrast, for lung fibrosis (evaluated in terms of Ashcroft score), a significant beneficial effect was observed only when MSC and CSD treatment were combined. Again, the improvement was similar whether MSCs from healthy or bleomycin-treated mice were used. This beneficial effect was further validated in IHC studies. While both CSD and MSC treatment greatly decreased ASMA levels in the bleomycin-treated lung, in combination they significantly increased inhibition of ASMA levels and improvement in tissue morphology/cellularity as compared to monotherapy with CSD or with MSCs.

### Example 2: Functional Subdomains of CSD

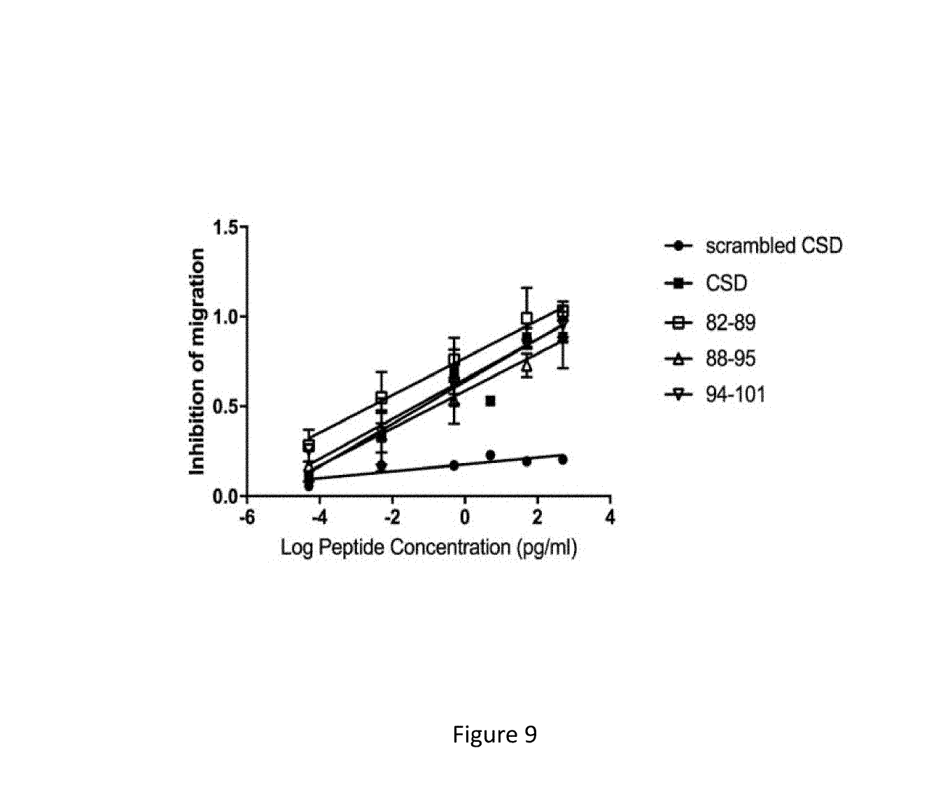

To study the structure-activity relationship of CSD to identify an optimal version to be developed for treating fibrotic diseases in human patients, subdomains of CSD were tested for their ability to inhibit the migration of human monocytes. Monocytes from healthy subjects were used which, when activated using TGFβ, migrate at an enhanced rate similar to monocytes from SSc patients (Tourkina et al., 2011, Fibrogenesis Tissue Repair, 4:15). TGFβ-induced migration was decreased essentially to the uninduced level by 50 pg/ml of CSD (FIG. 9). Substantial inhibition was observed at levels as low as 0.005 pg/ml. As expected, scrambled CSD gave little inhibition even at the highest dose tested. Like CSD, all three subdomains tested (Table 2) exhibited dose-dependent activity at extraordinarily low concentrations (FIG. 9).

TABLE 2
Exemplary CSD Sequences

| CSD Peptide or | | SEQ ID |
|---|---|---|
| Subdomain | Sequence | NO |
| Cav-1 AA 82-101 | DGIWKASFTTFTVTKYWFYR-NH2 | SEQ ID |
| (CSD) | | NO: 1 |
| Cav-1 AA 82-89 | DGIWKASF -NH2 | SEQ ID |
| | | NO: 2 |
| Cav-1 AA 88-95 | SFTTFTVT -NH2 | SEQ ID |
| | | NO: 3 |
| Cav-1 AA 94-101 | VTKYWFYR-NH2 | SEQ ID |
| | | NO: 4 |

Subdomains of CSD may have equivalent or better activity than full-length 20-aa CSD in inhibiting the fibrogenic differentiation of MSCs and promoting the adipogenic differentiation of MSCs. Shorter peptides may be less likely to have side effects and are easier to deliver.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A composition comprising a mesenchymal stem cell (MSC) and a caveolin-1 scaffolding domain (CSD) peptide or a subdomain, derivative, or analog thereof.

2. The composition of claim 1, wherein the CSD peptide or a subdomain, derivative, analog thereof comprises an amino acid sequence selected from the group consisting of SEQ ID NO:1, SEQ ID NO:2, SEQ ID NO:3, SEQ ID NO:4, and a combination thereof.

3. The composition of claim 1, wherein the MSC has the ability to differentiate into an adipocyte.

4. The composition of claim 1, wherein the MSC is autologous, allogeneic, syngeneic, or xenogeneic to a subject having fibrosis.

5. The composition of claim 4, wherein the fibrosis is scleroderma.

6. A composition comprising a treated MSC, wherein the treatment is selected from the group consisting of culture with a CSD peptide, culture with a subdomain of a CSD peptide, genetic modification with a nucleic acid molecule encoding a CSD peptide, and genetic modification with a nucleic acid molecule encoding a subdomain of a CSD peptide.

7. The composition of claim 6, wherein the CSD peptide or a subdomain of a CSD peptide comprises an amino acid sequence selected from the group consisting of SEQ ID NO:1, SEQ ID NO:2, SEQ ID NO:3, SEQ ID NO:4, and a combination thereof.

8. The composition of claim 6, wherein the MSC has the ability to differentiate into an adipocyte.

9. The composition of claim 6, wherein the MSC is autologous, allogeneic, syngeneic, or xenogeneic to a subject having fibrosis.

10. The composition of claim 9, wherein the fibrosis is scleroderma.

11. A method of treating fibrosis in a subject, the method comprising administering to a subject in need thereof an effective amount of a first composition comprising a MSC and an effective amount of a second composition comprising a CSD peptide or a subdomain, derivative, or analog thereof.

12. The method of claim 11, wherein the CSD peptide or a subdomain, derivative, analog thereof comprises an amino acid sequence selected from the group consisting of SEQ ID NO:1, SEQ ID NO:2, SEQ ID NO:3, SEQ ID NO:4, and a combination thereof.

13. The method of claim 11, wherein the first composition and second composition are administered concurrently.

14. The method of claim 11, wherein the first composition and second composition are administered at different times.

15. The method of claim 11, wherein the MSC is treated with a composition selected from the group consisting of a CSD peptide, a subdomain of a CSD peptide, a nucleic acid molecule encoding a CSD peptide, and a nucleic acid molecule encoding a subdomain of a CSD peptide.

16. The method of claim 15, wherein the MSC is treated prior to administration to the subject.

17. The method of claim 11, wherein one or more of the first and second composition are administered to the subject by a route selected from the group consisting local, subcutaneous, intravenous, oral, intramuscular, and a combination thereof.

18. The method of claim 11, wherein the MSC differentiates into an adipocyte in the subject.

19. The method of claim 11, wherein the MSC is autologous, allogeneic, syngeneic, or xenogeneic to a subject having fibrosis.

20. The method of claim 11, wherein the fibrosis is scleroderma.

21. A method of treating fibrosis in a subject, the method comprising administering to a subject in need thereof an effective amount of the composition of claim 1.

22. A method of treating fibrosis in a subject, the method comprising administering to a subject in need thereof an effective amount of the composition of claim 6.

23. The method of claim 22, wherein the MSC is treated prior to administration to the subject.

24. A kit comprising (a) a first composition comprising a MSC and (b) a second composition comprising a CSD peptide or a subdomain, derivative, or analog thereof.

25. The kit of claim 24, wherein the CSD peptide or a subdomain thereof comprises an amino acid sequence selected from the group consisting of SEQ ID NO:1, SEQ ID NO:2, SEQ ID NO:3, SEQ ID NO:4, or any combination thereof.

26. The kit of claim 24, wherein the MSC is treated with a composition selected from the group consisting of a CSD peptide, a subdomain of a CSD peptide, a nucleic acid molecule encoding a CSD peptide, and a nucleic acid molecule encoding a subdomain of a CSD peptide.


SEQUENCE LISTING (4 sequences)

SEQ ID NO: 1 - protein, 20 aa, Artificial Sequence
  Chemically synthesized
DGIWKASFTTFTVTKYWFYR

SEQ ID NO: 2 - protein, 8 aa, Artificial Sequence
  Chemically synthesized
DGIWKASF

SEQ ID NO: 3 - protein, 8 aa, Artificial Sequence
  Chemically synthesized
SFTTFTVT

SEQ ID NO: 4 - protein, 8 aa, Artificial Sequence
  Chemically synthesized
VTKYWFYR